(19) United States
(12) Patent Application Publication
(10) Pub. No.: US 2014/0228240 A1
(43) Pub. Date: Aug. 14, 2014

(54) Screening Blood for Protein Biomarkers and Uses Thereof in Alzheimer's Disease and Mild Cognitive Impairment

(71) Applicant: Emory University, Atlanta, GA (US)

(72) Inventor: William Hu, Atlanta, GA (US)

(73) Assignee: Emory University, Atlanta, GA (US)

(21) Appl. No.: 14/180,844

(22) Filed: Feb. 14, 2014

Related U.S. Application Data
(60) Provisional application No. 61/764,610, filed on Feb. 14, 2013.

(51) Int. Cl. G01N 33/543

(52) U.S. Cl. CPC G01N 33/54306
USPC 506/9; 506/39

(57) ABSTRACT

This disclosure is in the area of medical diagnostics that provides a method to assist in diagnosis and monitoring the progression of Alzheimer's disease and mild cognitive impairment (MCI).

### CROSS REFERENCE TO RELATED APPLICATIONS

This application claims priority to U.S. Provisional Patent Application 61/764,610 filed on Feb. 14, 2013, and is incorporated by reference in its entirety.

### STATEMENT REGARDING FEDERALLY FUNDED RESEARCH

This invention was made with government support under grants AG10124 and AG17585 awarded by National Institutes of Health. The government has certain rights in the invention.

### FIELD OF THE INVENTION

The invention is generally in the area of medical diagnostics. Certain embodiments provide systems and methods to assist in diagnosis and monitoring the progression of Alzheimer's disease (AD) and mild cognitive impairment (MCI) or other neurodegenerative diseases from a blood sample.

### BACKGROUND

Alzheimer's disease (AD) is a neurodegenerative disease that is marked by a progressive loss in memory, cognitive ability and altered behavior, with these symptoms eventually hampering the ability of an individual to perform relatively simple tasks. Up to 5.1 million Americans suffer from AD with estimates suggesting that by 2050 up to 16 million Americans will have the disease. Individuals with AD present amyloid plaques in the brain caused by the deposition of beta amyloid protein, a cleavage product of amyloid precursor protein (APP). The disease is also marked by a loss in connectivity between neurons in the brain and also intracellular filamentous fiber tangles referred to as neurofibriallary tangles. These tangles are composed of neurofilament and a hyperphosphorylated tau protein. Tau proteins are abundant in the CNS, and function in microtubule stabilization and flexibility in axons. Hyperphosphorylation of tau proteins results in microtubule destabilization.

When these alterations in the brain begin to develop, the individual is asymptomatic. Over time plaques and tangles accumulate and spread through the hippocampus and cerebral cortex. Both of these regions play essential roles in the maintenance of cognitive ability and memory. As the disease progresses, damage spreads leading to diminishing neuronal function and death of the affected neurons. Neuronal death is accompanied with brain tissue loss, eventually leading to the shrinking of the impacted regions, and ultimately shrinkage of the brain. These pathological characteristics of Alzheimer's disease are associated with both late-onset and inherited early onset forms of AD.

Four genes, namely apolipoprotein E (apoE), amyloid precursor protein (APP), presenilin 1 (PS 1) and presenilin 2 (PS2) have been linked to early onset AD. APP, PS1, and PS2 have been found to directly cause AD. Presenilin proteins modulate the proteolytic activity of gamma secretase. These enzymes are responsible for the cleavage of APP, and production of beta-amyloid. Mutation or over-expression of APP, PS1, and PS2 results in an increase in beta amyloid, and the accumulation of beta amyloid deposits in the brain. ApoE catalyzes the clearance of beta-amyloid. Genetic variations of apoE can lead to the accumulation of beta amyloid and plaque. Therefore, allele frequency of apoE has been shown to be a strong genetic indicator for AD.

Clinically, symptoms of AD are cognitive loss including impaired reasoning, loss of memory, confusion, difficulty learning new things, difficulty with problem solving, difficulty recognizing family or friends, language impairment, visual and spatial issues. This dementia can also be warning signs for mild cognitive impairment (MCI). Individuals with this condition have memory problems however the symptoms for MCI are not as severe as those associated with AD. Recent reports suggest that older individuals with MCI are likely to develop AD. Changes in behavior and personality are also symptoms of AD.

By the time an individual with AD becomes symptomatic, tangles and plaques have already begun to form in regions of the brain that control learning, memory, and thinking Therefore, it would be beneficial to detect MCI and AD early, before the individual becomes symptomatic. Current methods of early detection include cerebral amyloid imaging or removal of cerebrospinal (CSF) to determine levels of established biomarkers associated with AD. These diagnostic measures are typically more invasive and expensive.

Therefore, it is an object of the invention to provide blood based biomarkers that are associated with, indicative of, or predictive of a subject's likelihood of developing Alzheimer's disease, mild cognitive impairment and other neurodegenerative diseases.

It is another object of the invention to provide alternative or complementary methods for diagnosing and monitoring the progression of Alzheimer's disease, mild cognitive impairment and other neurodegenerative diseases.

It is a further objection of the invention to provide systems and methods of diagnosing and monitoring that are minimally invasive, inexpensive, and highly specific.

### SUMMARY

Methods of determining if a subject has Alzheimer's disease (AD) or Mild Cognitive Impairment (MCI) or is at risk of developing AD or MCI are provided. The methods typically include measuring the protein levels of Apolipoprotein E (apoE); B-type natriuretic peptide; C-reactive protein; and pancreatic polypeptide in a blood sample from the subject, and determining that the subject has Alzheimer's disease (AD) or Mild Cognitive Impairment (MCI), or is at risk of developing AD or MCI when one or more of the following first conditions: (i) the level of apoE in the subject's blood sample is reduced relative to an apoE control value; (ii) the level of B-type natriuretic peptide in the subject's blood sample is increased relative to a B-type natriuretic peptide control value; (iii) the level of C-reactive protein in the subject's blood sample is reduced relative to a C-reactive protein control value; (iv) the level of pancreatic polypeptide in the subject's blood sample is increased relative to a pancreatic polypeptide control value; are met.

In some embodiments, the methods optionally include measuring the protein levels of Cortisol, FAS, IL-3, IL-10, IL-12p40, IL-13, IL-15, Osteopontin, Resistin, Stem cell factor, E-selectin, serum amyloid protein, or any combination thereof in the blood sample from the subject; and determining that the subject has Alzheimer's disease (AD) or Mild Cognitive Impairment (MCI), or is at risk of developing AD or MCI when one or more of the second conditions: (i) the level of Cortisol in the subject's blood sample is increased relative to a Cortisol control value; (ii) the level of FAS in the subject's blood sample is increased relative to a FAS control value; (iii) the level of IL-3 protein in the subject's blood sample is increased relative to an IL-3 control value; (iv) the level of IL-10 in the subject's blood sample is increased relative to an IL-10 control value; (v) the level of IL-12p40 in the subject's blood sample is increased relative to an IL-12p40 control value; (vi) the level of IL-13 in the subject's blood sample is increased relative to an IL-13 control value; (vii) the level of IL-15 in the subject's blood sample is increased relative to an IL-15 control value; (viii) the level of Osteopontin in the subject's blood sample is increased relative to an Osteopontin control value; (ix) the level of Resistin in the subject's blood sample is increased relative to a Resistin control value; (x) the level of Stem cell factor in the subject's blood sample is increased relative to a Stem cell factor control value; (xi) the level of E-selectin in the subject's blood sample is reduced relative to an E-selectin control value; (xii) the level of serum amyloid protein in the subject's blood sample is reduced relative to a serum amyloid protein control value; are met.

Methods of determining the efficacy of a treatment for AD or MCI are also provided. The methods typically include measuring the protein levels of Apolipoprotein E (apoE); B-type natriuretic peptide; C-reactive protein; and pancreatic polypeptide in a second blood sample from a subject with AD or MCI undergoing a treatment for AD or MCI, wherein the second blood sample is obtained from the subject after a sufficient amount of time has passed for the treatment to reduce one or more symptoms of the AD or MCI; and determining that the treatment is effective for treating AD or MCI when one or more of the following first conditions: (i) the level of apoE in the subject's second blood sample is increased relative to a first blood sample taken from the subject prior the treatment; (ii) the level of B-type natriuretic peptide in the subject's second blood sample is decreased relative to a first blood sample taken from the subject prior the treatment; (iii) the level of C-reactive protein in the subject's second blood sample is increased relative to a first blood sample taken from the subject prior the treatment; (iv) the level of pancreatic polypeptide in the subject's second blood sample is decreased relative to a first blood sample taken from the subject prior the treatment; are met.

The methods can optionally include measuring the protein levels of one or more of Cortisol, FAS, IL-3, IL-10, IL-12p40, IL-13, IL-15, Osteopontin, Resistin, Stem cell factor, E-selectin, serum amyloid protein, or any combination thereof in the second blood sample from the subject; and determining that the treatment is effective for treating AD or MCI when one or more of the following second conditions: (i) the level of Cortisol in the subject's second blood sample is decreased relative to a first blood sample taken from the subject prior the treatment; (ii) the level of FAS in the subject's second blood sample is decreased relative to a first blood sample taken from the subject prior the treatment; (iii) the level of IL-3 protein in the subject's second blood sample is decreased relative to a first blood sample taken from the subject prior the treatment; (iv) the level of IL-10 in the subject's second blood sample is decreased relative to a first blood sample taken from the subject prior the treatment; (v) the level of IL-12p40 in the subject's second blood sample is decreased relative to a first blood sample taken from the subject prior the treatment; (vi) the level of IL-13 in the subject's second blood sample is decreased relative to a first blood sample taken from the subject prior the treatment; (vii) the level of IL-15 in the subject's second blood sample is decreased relative to a first blood sample taken from the subject prior the treatment; (viii) the level of Osteopontin in the subject's second blood sample is decreased relative to a first blood sample taken from the subject prior the treatment; (ix) the level of Resistin in the subject's second blood sample is decreased relative to a first blood sample taken from the subject prior the treatment; (x) the level of Stem cell factor in the subject's second blood sample is decreased relative to a first blood sample taken from the subject prior the treatment; (xi) the level of E-selectin in the subject's second blood sample is increased relative to a first blood sample taken from the subject prior the treatment; (xii) the level of serum amyloid protein in the subject's second blood sample is increased relative to a first blood sample taken from the subject prior the treatment; are met.

In preferred embodiments of the disclosed methods, two, three or all four of the first conditions are met. In some embodiments, 1, 2, 3, 4, 5, 6, 7, 8, 9, 10, 11, or all 12 of the second conditions are met. In some embodiments, the method has a sensitive of at least 70%, 75%, 80%, 85%, 90%, 95%, 96%, 97%, 98%, 99%, or more than 99%.

The samples can be whole blood, plasma, or serum. In preferred embodiments, the protein levels are measured with an immunoassay. The immunoassay can be bead-based. For measuring more than one protein level, the assay is preferably a multiplex assay. In some embodiments the determining step or steps is carried our using a computational system.

In some embodiments, the protein levels are determined to be increased or decreased when the p value between the protein level and the corresponding control value is less than 0.1, preferably less than 0.05, more preferably less than 0.01. The control value for a protein can be the measurement of the protein level in a blood sample from a control subject that does not have AD or MCI, or an average value for two or more control subjects that do not have AD or MCI. In some embodiments the control subjects score 26 or greater on the mini-mental state examination (MMSE). In some embodiments, the control subjects were determined not to have AD or MCI based on measuring β-amyloid 1-42 (Aβ42), total tau (t-tau), t-tau/Aβ42 ratio or combination thereof in their cerebral spinal fluid.

In certain embodiments, this disclosure relates to a panel of protein levels that is altered in the blood of individuals with Alzheimer's disease (AD) and mild cognitive impairment (MCI). This panel of proteins found to be altered in the blood can be used to assess the progression or assist in the diagnosis of MCI and AD. This disclosure provides a method for the measurement of these proteins in the blood, the comparison of these measurements with reference levels of each protein, and determining whether the subject is at risk of Alzheimer's disease (AD) and Mild Cognitive Impairment (MCI); wherein if the subject has altered levels of the protein biomarkers this indicates a risk of Alzheimer's disease (AD) or Mild Cognitive Impairment (MCI). This analysis can be performed using the protein biomarkers individually or collectively. Data obtained from this analysis can then be used to then diagnose or assess the progression of these neurodegenerative disorders.

In certain embodiments, the disclosure relates to methods to assist in the diagnosis or progression or identifying a candidate agent for treatment of Alzheimer's disease (AD) and Mild Cognitive Impairment (MCI) or other neurological disease including the steps of, a) measuring a blood sample from a subject for levels of the following proteins Apolipoprotein E (apoE); B-type natriuretic peptide; C-reactive protein; pancreatic polypeptide; and at least five more the following proteins, Cortisol, E-selectin, FAS, Gamma-IFN-induced monokine, IL-3, IL-10, IL-12p40, IL-13, IL-15, Osteopontin, Resistin, Serum amyloid protein, and Stem cell factor; providing measured levels; b) comparing the normalized measured levels of proteins with reference levels wherein the reference levels are obtained from normalized measured values; c) determining whether the subject is at increased risk of Alzheimer's disease (AD) and Mild Cognitive Impairment (MCI); wherein if the subject has altered levels of the proteins compared to reference levels this indicates an increased risk of Alzheimer's disease (AD) or Mild Cognitive Impairment (MCI).

In certain embodiments at least six, seven, eight, nine, ten, eleven, twelve, thirteen, or more proteins are measured with apoE, BNP, CRP, and pancreatic polypeptide.

In certain embodiments, wherein if the subject has normal levels of the proteins compared to reference levels this indicates an cognitive impairment not associated with Alzheimer's disease (AD) such as Parkinson's disease or dementia with Lewy bodies.

In certain embodiments, the method further includes the steps of testing the subject for β-amyloid 1-42 (Aβ42), total tau (t-tau) and t-tau/Aβ42 ratio from a CSF sample provided from the subject is indicated to have a risk of Alzheimer's disease (AD) and Mild Cognitive Impairment (MCI) based on the measured levels of the proteins.

In certain embodiments, the diagnosis of AD is aided by determining a difference between the normalized measured levels of proteins to the reference levels of the protein from non-AD samples wherein the difference meets or exceeds a statistically significant difference between normalized measured values of proteins in the blood samples from individuals without AD and individuals with AD, wherein the statistically significant difference indicates a diagnosis of AD.

In certain embodiments, the disclosure relates to method for diagnosing or monitoring the progression of AD or MCI by obtaining a measured value for apoE, BNP, CRP, and pancreatic polypeptide in blood sample; and comparing said measure value of apoE, BNP, CRP, and pancreatic polypeptide with a reference value; wherein the measured level of BNP and Pancreatic polypeptide increases, wherein the measured levels of apoE and CRP decrease indicates a diagnosis or the progression of MCI or AD.

In certain embodiments, the method further includes comparing measured values from blood samples for BNP, Cortisol, FAS, IL-3, IL-10, IL-12p40, IL-13, IL-15, Osteopontin, Pancreatic polypeptide, Resistin, Stem cell factor, ApoE, CRP, E-selectin, and serum amyloid protein with reference values for BNP, Cortisol, FAS, IL-3, IL-10, IL-12p40, IL-13, IL-15, Osteopontin, Pancreatic polypeptide, Resistin, Stem cell factor, apoE, CRP, E-selectin, and serum amyloid protein, wherein measured values are from individuals with an MMSE score less than 26, wherein the measured value for ApoE, CRP, E-selectin, and serum amyloid protein decreases, wherein measured values for BNP, Cortisol, FAS, IL-3, IL-10, IL-12p40, IL-13, IL-15, Osteopontin, Pancreatic polypeptide, Resistin, Stem cell factor increase indicating cognitive impairment such as MCI or AD.

In certain embodiments, the subject is a human subject at risk of, exhibiting symptoms, and/or seeking a diagnosis for AD. Typically, sample is a whole blood, plasma or serum.

In certain embodiments, the measuring includes mixing the sample with a solid surface including a ligand or capture antibody to the protein and detecting the protein bound to the surface.

In certain embodiments, the reference levels for the proteins are obtained by a method including: determining the mean value of the normalized measured levels of the protein biomarkers in normal individuals with Mini Mental State examination (MMSE) scores from 26-30, having statistically significant difference from the mean value of the normalized measured levels of the proteins from a subjects with MMSE score of lower than 24.

In certain embodiments, the significant difference in the normalized measured values of the 17 protein biomarkers in the blood samples from individuals with AD in comparison to samples from individuals without AD is calculated using Significance Analysis of Microarrays (SAM).

In certain embodiments, the statistical difference in the normalized measured values of the 17 protein biomarkers as determined by SAM has a p-value range from 0.001 to 0.822.

In certain embodiments, the normalized measured value is determined by normalizing it relative to the median values of protein biomarker levels from individuals with and without AD.

In certain embodiments, the measured levels comprise methods selected from this group consisting of SPSS 17.0, Significance Analysis of Microarrays, PASS11, and Intersection Union Test.

In certain embodiments, determining the statistically significant difference associated with a diagnosis of AD includes: calculating the mean value of normalized measured values of each of at least the seventeen protein biomarkers in the blood samples from individuals with AD; calculating a mean value of normalized measured values of each of at least seventeen protein biomarkers in the blood samples from a group of individuals without AD; wherein the individuals from both groups are in the same age group; and finding a statistically significant difference between the mean values of the normalized measured values of the at least seventeen protein biomarkers in the blood samples between the two groups.

In certain embodiments, determining the significant difference associated with the progression of AD includes: calculating the mean value of normalized measured values of each of at least the seventeen protein biomarkers in the blood samples from individuals with AD; calculating a mean value of normalized measured values of each of at least seventeen protein biomarkers in the blood samples from a group of individuals without AD; wherein the individuals from both groups are in the same age group; and finding a statistically significant difference between the mean values of the normalized measured values of the at least seventeen protein biomarkers in the blood samples between the two groups.

In certain embodiments, the disclosure relates to kits including at least one reagent specific for at least one protein selected from the group consisting of proteins disclosed herein and instructions for carrying out the methods disclosed herein. In certain embodiments, the reagent is specific for at least four, five, six, seven, eight, nine, ten, eleven, twelve, thirteen, fourteen, fifteen or more proteins disclosed herein.

In certain embodiments, the reagent specific for a protein is an antibody, or fragment thereof, with affinity for said protein. In certain embodiments, the surface further includes a blood sample. In certain embodiments, the blood sample is from a subject at risk of or exhibiting symptoms of cognitive impairment including: APOE4 allele number, an MMSE score lower than 24, Clinical Dementia Rating (CDR) of 0.5-1, alteration in β-amyloid 1-42 (Aβ42), total tau (t-tau) or t-tau/Aβ42 ratio from a CSF sample.

In certain embodiments, the disclosure relates to a surface including attached thereto, at least one reagent specific for each protein as provided herein. In certain embodiments, the surface further includes the reagent bound to the protein and a secondary reagent specific for the protein bound to the protein wherein the secondary agent includes a marker. In certain embodiments the marker is a fluorescent molecule or reporter.

In certain embodiments, the subject is at risk of cognitive impairment because they are at least 50, 60, or 65 years old.

In certain embodiments, methods disclosed herein further comprise the step of obtaining a value for the comparison of the measured level to the reference level and recording the value on a computer readable medium or format. In certain embodiments, the computer records the values associated with the protein levels and uses an algorithm to assign a value for risk of AD or MCI that ranges between a number scale, such as likelihood of having AD or MCI on a scale of one to ten wherein one is very unlikely, and ten is very likely. In certain embodiments, the algorithm determines relative number of markers that are high or lower than the reference levels, and provides a higher risk for a higher number of markers outside the reference levels, a lower risk for a low number of markers outside the reference levels or a combination thereof.

In certain embodiments, the disclosure relates to a system for detecting proteins discloses herein including a solid surface and a visualization device.

### BRIEF DESCRIPTION OF THE DRAWINGS

FIG. 1 depicts a flow diagram which describes the subjects included in a study that focused on identifying protein biomarkers that were predictive of MCI and AD. Participants from the University of Pennsylvania and the Alzheimer's Disease Neuroimaging Initiative (ADNI) were stratified into normal cognition, mild cognitive impairment (MCI) and clinical probable Alzheimer's disease. Participants form Washington University were characterized according to Clinical Dementia Rating scale (CDR) with 0 meaning the individual had normal cognitive function, 0.5 indicating the individual had MCI, and 1 indicating the individual had AD.

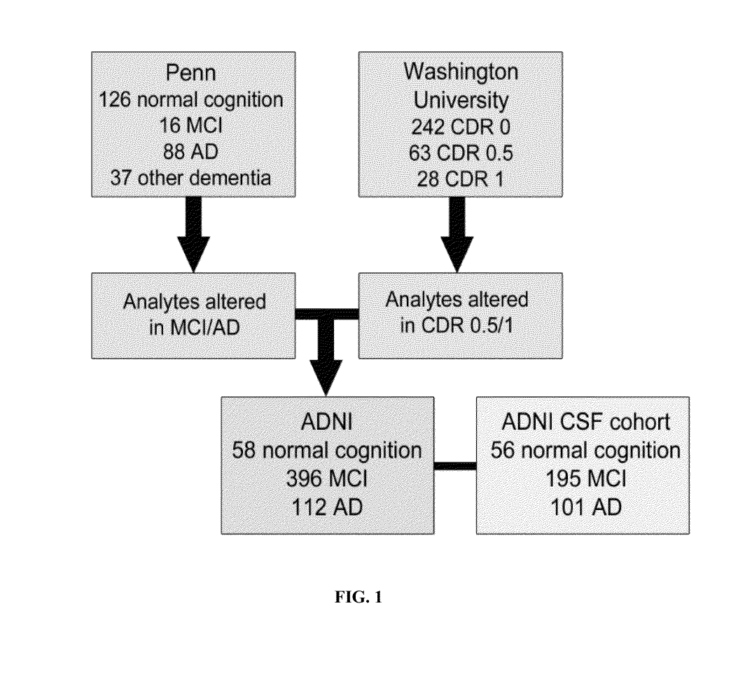

FIG. 2 shows an example of a system configured to measure protein biomarkers with a visual device.

FIG. 3 is a graphical representation of the AD score of a population of subjects screened for AD and cognitive impairment. Black circles represent subjects having Alzheimer's Disease, grey circles represent subjects having mild cognitive impairment, and white circles represent healthy control subjects.

### DETAILED DESCRIPTION

### I. Definitions

As used herein, methods for “aiding diagnosis” or “assisting in diagnosis” both refer to methods that assist in making a clinical determination regarding the presence or progression of the AD or MCI, and may or may not be conclusive with respect to the definitive diagnosis.

As used herein, the term “predicting” refers to making a finding with notably enhanced likelihood of developing MCI or AD.

As used herein, “blood sample” or “whole blood sample” encompasses a biological sample which is derived from blood obtained from an individual and can be used in a diagnostic or monitoring assay. The definition encompasses blood, plasma, and serum.

As used herein, a “reference value” can be an absolute value; a relative value; an average value; a median value, a mean value, or a value as compared to a particular control or baseline value. A reference value can be based on an individual sample or a large number of samples, such as from AD patients or normal individuals.

A “normalized measured” value refers to a measurement taken and adjusted to take background into consideration. Background subtraction to obtain total fluorescence is considered a normalized measurement. The background subtraction allows for the correction of background fluorescence that is inherent in the optical system and assay buffers.

A “log transformed” value refers to a measurement taken and adjusted to take non-normal distribution into consideration. Log transformation allows for the correction of skewed distribution for one or more of the protein levels.

A “Z-transformed” value refers to a measure taken and adjusted to take batch-to-batch variations in protein measurements into account. Z-transformation allows for correction of batch-level differences and comparison of protein measurements obtained on different days and using different reagent lots.

### II. Biomarkers for Multi-analyte Profiling

Clinical diagnosis of mild cognitive impairment (MCI) and probable Alzheimer disease (AD) is increasingly aided by biomarkers predictive of underlying pathology (Shaw et al., Ann Neurol, 2009, 65:403-413; Fagan et al., Arch Neurol 2007; 64:343-349; Perrin et al., Nature, 2009, 461: 916; Kondziella et al., NeuroReport 2009, 20:825-827; Kerola et al., Ann Med, 2010, 42:207-215).

These include:

1) CSF biomarkers reflecting the plaque and tangle pathology underlying AD such as -amyloid 1-42 (Aβ42), total tau (t-tau), and tau phosphorylated at threonine 181 (p-tau181);

2) substrate specific brain imaging such as 11C and 18F PET imaging; and

3) structural MRI findings such as hippocampal volume.

However, each modality has a different sensitivity-specificity profile, and additional technical barriers and patient preferences may dictate the successful implementation of any biomarker into clinical practice, including aversion to having a lumbar puncture for CSF biomarkers and cost for advanced imaging. Thus, a blood-based test is an appealing alternative because of its simplicity and cost-effectiveness for widespread clinical use as well as in specialty centers.

A number of studies have generated enthusiasm for a blood-based test predictive of underlying AD pathology. See, for example, Hu et al., Neurology 2012, 79:897-905; Ray et al., Nat Med, 2007, 13(11):1359-1362, U.S. Pat. No. 7,598,049, and Reddy et al., Cell 2011; 144:132-142. However, some previous serum analytes predictive of AD were found to have only modest accuracy in plasma. See O'Bryant et al., PloS one, 2011, 6:e28092.

Therefore, improved methods and systems for blood-based diagnosis and assessment of mild cognitive impairment (MCI), Alzheimer's Disease (AD) and other dementias are disclosed. Multi-analyte profiling approaches to plasma proteins and peptides, such as those describe herein, can also yield biologically important signatures of disease and endophenotypes to allow for prognostication and therapeutic development.

The Examples below describe the results of experiments designed to identify blood biomarkers that can be used in the diagnosis and assessment of mild cognitive impairment (MCI), Alzheimer's Disease (AD) and other dementias. Overlapping plasma analytes associated with MCI/AD in 2 independently recruited and characterized cohorts were identified and included Cortisol; E-selectin; FAS; Gamma-IFN-induced monokine; IL-3; IL-10; IL-12p40; IL-13; IL-15; Osteopontin; Resistin; Serum amyloid protein; Stem cell factor; Apolipoprotein E (apoE); B-type natriuretic peptide; C-reactive protein; and pancreatic polypeptide. Therefore, the methods, assays, and systems described herein typically include analyzing expression levels of one or more of the biomarkers: Cortisol; E-selectin; FAS; Gamma-IFN-induced monokine; IL-3; IL-10; IL-12p40; IL-13; IL-15; Osteopontin; Resistin; Serum amyloid protein; Stem cell factor; Apolipoprotein E (apoE); B-type natriuretic peptide; C-reactive protein; and pancreatic polypeptide, preferably in a blood sample, more preferably in a plasma sample from a subject.

The correlation between plasma analytes associated with MCI/AD and CSF AD biomarker levels were then validated by utilizing an independent cohort of 566 participants from the multicenter Alzheimer's Disease Neuroimaging Initiative (ADNI), involving subjects with both CSF biomarkers and blood plasma analyte levels.

The Examples below illustrate that among the identified biomarkers, changes in apoE, BNP, CRP, and pancreatic polypeptide levels were associated with MCI/AD diagnosis and CSF AD biomarker profiles in ADNI. Therefore, the methods, assays, and systems described herein preferably include analyzing expression levels of one or more; preferably two or more; more preferably three or more; most preferably all four of the biomarkers: apoE, BNP, CRP, and pancreatic polypeptide. In the most preferred embodiments, the methods, assays, and systems described herein include analyzing expression levels of one, two, three, or all four of apoE, BNP, CRP, and pancreatic polypeptide in combination with 1, 2, 3, 4, 5, 6, 7, 8, 9, 10, 11, 12, or all 13 of Cortisol; E-selectin; FAS; Gamma-IFN-induced monokine; IL-3; IL-10; IL-12p40; IL-13; IL-15; Osteopontin; Resistin; Serum amyloid protein; Stem cell factor.

The Examples also describe logistic regression analysis performed with dichotomous outcome as dependent variables, including “CSF Aβ42<193 pg/mL”, “t-Tau>91 pg/mL”, and “t-Tau/Aβ42 ratio>0.39”. These values have strong association with AD. The results indicated that a diagnostic panel including both demographic variables (age, gender, education, presence of APOE ε4 allele) and plasma analytes disclosed herein was much more sensitive in detecting abnormal CSF Aβ42 levels than a panel consisting of demographic variables alone (85% vs. 69%, p<0.0001).

Along with the known association between apoE genotyping and CSF AD biomarker levels, these AD plasma biomarkers also correlated with CSF Aβ42 levels and t-tau/Aβ42 ratios. These plasma AD biomarkers help predict underlying AD pathology through their relationships to established CSF biomarkers of AD, and therefore support a conclusion that they can serve as the basis of a plasma-based screen for AD.

Among the preferred plasma AD biomarkers disclosed herein, apoE, BNP, CRP, and pancreatic polypeptide levels were associated with CSF Aβ42 levels and t-tau/Aβ42 ratios. BNP is a marker of left ventricular dysfunction, and is elevated in acute strokes and vascular dementia. Elevated BNP levels are also associated with cognitive decline in vascular disease and the development of AD and vascular dementia (independent of heart failure). Elevated BNP may thus reflect shared risk factors between heart failure and AD or an unknown step in AD pathogenesis.

Pancreatic polypeptide levels were previously identified by TARC. Pancreatic polypeptide is a small signaling peptide associated with postprandial appetite suppression present in multiple brain regions, including those affected by AD such as the hippocampus and locus ceruleus. Increased plasma pancreatic polypeptide levels could reflect impaired transport across the blood-brain/CSF barrier through yet unclear mechanisms, but it is also elevated in the CSF of patients with AD. Similar changes in patients with non-AD dementia further suggest that elevated pancreatic polypeptide levels may reflect neuronal loss irrespective of etiology, although such elevated levels can still serve as a potential plasma marker of neuronal injury.

Similarly, CRP was not specifically associated with CSF Aβ42 levels or t-tau/Aβ42 ratios, although it complemented BNP and pancreatic polypeptides in predicting CSF AD biomarkers. In other studies, CRP has been found to be decreased, increased, or unchanged in AD. Alterations in CRP levels may again reflect neuronal injury, although its levels may be more susceptible to patient selection and endophenotypes than other plasma biomarkers.

### III. Methods of Diagnosis and Assessment

A. Determining Biomarker Levels

Methods of assisting in the diagnosis of, monitoring the progression of, or identifying candidate agents for treatment of Alzheimer's disease (AD), Mild Cognitive Impairment (MCI), and other cognitive and neurodegenerative disorders are provided. The methods typically include obtaining a measured valve for one of more of the biomarkers disclosed herein. In some embodiments the measured value is obtained by measuring or extrapolating the level of the protein biomarkers in a sample obtained from the subject. The blood sample can be derived from whole blood, serum or plasma. Next, the measured level(s) is compared to a reference value to determine if the subject has AD, MCI, or another cognitive or neurodegenerative disorder or if the subject is likely to develop AD, MCI, or another cognitive or neurodegenerative disorder. Methods of measuring protein biomarker levels are discussed in more detail below.

In preferred embodiments at least one, preferable two, more preferably three, most preferably all four of Apolipoprotein E (apoE); B-type natriuretic peptide; C-reactive protein; and pancreatic polypeptide are measured and compared. In some embodiments, 1, 2, 3, 4, 5, 6, 7, 8, 9, 10, 11, 12, or all 13 of Cortisol; E-selectin; FAS; Gamma-IFN-induced monokine; IL-3; IL-10; IL-12p40; IL-13; IL-15; Osteopontin; Resistin; Serum amyloid protein; and Stem cell factor are measured and compared to reference levels.

Typically, increasing the number of biomarkers that are measured and compared can increase the accuracy or sensitivity for the test for diagnosing Alzheimer's disease (AD), Mild Cognitive Impairment (MCI), and other cognitive and neurodegenerative disorders are provided. For example, Apolipoprotein E (apoE); B-type natriuretic peptide; C-reactive protein; and pancreatic polypeptide, when measured and compared in parallel, are effective for diagnosing AD with a sensitivity of 70% or greater. Adding additional analysis of additional biomarkers in parallel can further increase the sensitivity of the diagnosis. In some embodiments, the methods disclosed herein have a sensitivity of 70%, 75%, 80%, 85%, 90%, 95%, 96%, 97%, 98%, 99%, or greater than 99% for diagnosing AD, MCI, or another cognitive or neurodegenerative disorder or determining if a subject is likely to develop AD, MCI, or another cognitive or neurodegenerative disorder. Therefore, in some embodiments, measured levels are obtained for at least, four, five, six, seven eight, nine, ten, eleven, twelve, thirteen, fourteen, fifteen, or more protein biomarkers, and preferably including Apolipoprotein E (apoE); B-type natriuretic peptide; C-reactive protein; and pancreatic polypeptide.

In some embodiments, the methods include obtaining the measured levels of β-amyloid 1-42 (Aβ42), total tau (t-tau) and/or determining t-tau/Aβ42 ratio in cerebral spinal fluid samples for obtained from the subject. Therefore, the measured levels of the protein biomarkers from the blood and the measured levels of the CSF biomarkers are considered in combination to determine if the subject has, or is at risk for developing AD, MCI, or other cognitive or neurodegenerative diseases.

In addition this disclosure has identified methods of characterizing AD and MCI patients by obtaining measured values for apoE, BNP, CRP, and pancreatic polypeptide from blood samples. Alterations in apoE, BNP, CRP, and pancreatic polypeptide levels are strongly associated with pathological symptoms of AD and MCI, specifically CSF Aβ42 levels and t-tau/Aβ42. The information thus obtained may be used to aid in stratification of diagnosis of MCI or AD.

To derive a risk for AD and MCI, levels of apoE, BNP, CRP, and pancreatic polypeptide as well as 1, 2, 3, 4, 5, 6, 7, 8, 9, 10, 11, 12, or all 13 of Cortisol; E-selectin; FAS; Gamma-IFN-induced monokine; IL-3; IL-10; IL-12p40; IL-13; IL-15; Osteopontin; Resistin; Serum amyloid protein; and Stem cell factor are measured. Protein levels are log-transformed to achieve normal distribution. In addition, Z-transformation was applied to 6 analytes (CRP, E selectin, Fas, IL-3, IL-13, and pancreatic polypeptide). Adjusted protein levels are then analyzed using an artificial neural network taking into account age and gender designed to maximize sensitivity as a screening test for AD and MCI. The artificial neural network is developed by cross-validation and cross-training of existing subjects with normal cognition, MCI, or AD, and generating a probability score of MCI/AD. The probability is translated into an AD Score for each subject, with 50 being the threshold beyond which the subject is at increased risk for MCI/AD. This threshold can achieve 88% sensitivity to detect AD and 75% sensitivity to detect MCI, and considers 39% of healthy seniors as at risk for MCI/AD. For each prospective subject undergoing this analysis, measured and transformed proteins levels will be entered into this model with age and gender to generate an AD score.

Another risk score is generated by analysis using support vector machine designed to maximize sensitivity. In this analysis, apoE, BNP, CRP, and pancreatic polypeptide levels are represented as functions of age and gender, and an optimal division to maximize sensitivity for MCI/AD while minimizing overall classification inaccuracy is derived for each protein by a non-linear function. A hyperplane is created by combining all these non-linear functions, and the expansion of the high dimensional space and hyperplane is achieved by adding 1, 2, 3, 4, 5, 6, 7, 8, 9, 10, 11, 12, or all 13 of Cortisol; E-selectin; FAS; Gamma-IFN-induced monokine; IL-3; IL-10; IL-12p40; IL-13; IL-15; Osteopontin; Resistin; Serum amyloid protein; and Stem cell factor. The final hyperplane represents the threshold at which a subject is at high risk for MCI/AD, and the AD score is generated from the distance from this hyperplane (increasing likelihood with increasing distance from the hyperplane). For each prospective subject undergoing this analysis, measured and transformed proteins levels will be entered into this model with age and gender to generate an AD score.

In addition to identifying relative AD risk, an important element of this disclosure is informing physicians and/or patients with higher relative risk what potential interventions could be taken. This is an important aspect of this disclosure, as currently there is a pervasive sense of therapeutic nihilism in the AD community. Thus, the disclosed methods to identify AD risk with a specific interpretive report that couples interventions to risk variables are a significant improvement to the field. The importance of the identification of these proteins is that they be modifiable by pharmacological and/or dietary based interventions in a biomarker specific fashion (see examples below) and can be assessed recurrently to ascertain whether said interventions are having their intended effect.

1) Pancreatic Polypeptide:

Amylin (pancreatic polypeptide) and amyloid-beta (Aβ) protein, which are deposited within pancreatic islets of diabetics and brains of Alzheimer's patients respectively, share many biophysical and physiological properties. Emerging evidence indicates that the amylin receptor is a putative target receptor for the actions of human amylin and Aβ in the brain. The amylin receptor consists of the calcitonin receptor dimerized with a receptor activity-modifying protein and is widely distributed within central nervous system. Both amylin and Aβ directly activate this G protein-coupled receptor and trigger multiple common intracellular signal transduction pathways that can culminate in apoptotic cell death. Moreover, amylin receptor antagonists can block both the biological and neurotoxic effects of human amylin and Aft Amylin receptors thus appear to be involved in the pathophysiology of Alzheimer's disease and diabetes, and could serve as a molecular link between the two conditions that are associated epidemiologically.

Misfolded human islet amyloid polypeptide (PP) in pancreatic islets is associated with the loss of insulin-secreting beta cells in type 2 diabetes. Diet and exercise have been shown to reduce pancreatic polypeptide and may mitigate AD risk. Subjects with T2D who received 24 weeks of diet combined with aerobic exercise were examined at weeks 0, 12 and 24. β-cell function was assessed and changes in glucose sensitivity correlated negatively with changes in plasma concentrations of PP, both in fasting and during hyperinsulinemia.

Insulin secretion impairment and cell apoptosis may be due to mitochondrial dysfunction in pancreatic beta cells. Attenuation of mitochondrial dysfunction provides a mechanism of potential intervention or prevention in AD.

Nicotinamide riboside improves mitochondrial function. Nicotinamide adenine dinucleotide (NAD)(+), is a coenzyme involved in redox activities in the mitochondrial electron transport chain—and the activation of NAD(+) expression has been linked with a decrease in beta-amyloid (Aβ) toxicity in Alzheimer's disease (AD). Nicotinamide riboside (NR) is a NAD(+) precursor, promotes peroxisome proliferator-activated receptor-γ coactivator 1 (PGC)-1α expression in the brain. Evidence has shown that PGC-1α is a crucial regulator of Aβ generation because it affects β-secretase (BACE1) degradation. dietary treatment with NR might benefit AD cognitive function and synaptic plasticity, in part by promoting PGC-1α-mediated BACE1 ubiquitination and degradation, thus preventing Aβ production in the brain.

2) BNP

Several traditional cardiovascular risk factors have been associated with the risk of dementia. New evidence suggests that the brain renin angiotensin system has two opposing pathways: a damaging pathway and a neuro-protective pathway. Both pathways are involved in the amyloid hypothesis (Aβ cascades) and vascular mechanisms of Alzheimer's disease.

Treatment with ARBs, useful in the treatment of hypertension and CHF, lower BNP levels, and have been hypothesized to be neuroprotective. Telmisartan, an angiotensin (Ang) II type I receptor blocker (ARB), results in a significant reduction of the plasma brain natriuretic peptide level infiltration of macrophages, and inhibits the activation of matrix metalloproteinases-2 and -9 (MMPs-2/9),

Several studies have suggested that ARBs have cognitive protective effects that are related to their ability to decrease production and oligomerization and increase degradation of Aβ and their vascular effects (improve blood-brain barrier, restore endothelial function, decrease inflammation, and increase cerebral blood flow). Human observational studies have further suggested that ARB use is associated with decreased risk of Alzheimer's disease and protection against future cognitive decline. ARB use is associated with decreased amyloid deposition in the brain in Alzheimer's disease and can provide potential cognitive protection in those with mild cognitive impairment, a prodromal state for Alzheimer's disease, and dementia, especially those with co morbid hypertension.

There is epidemiological and experimental evidence for involvement of cholesterol metabolism in the development and progression of Alzheimer disease. ApoE regulates lipid homeostasis by mediating lipid transport from one tissue or cell type to another. In peripheral tissues, ApoE is primarily produced by the liver and macrophages, and mediates cholesterol metabolism in an isoform-dependent manner. ApoE4 is associated with hyperlipidemia and hypercholesterolemia, which lead to atherosclerosis, coronary heart disease and stroke.

In the CNS, ApoE is mainly produced by astrocytes, and transports cholesterol to neurons via ApoE receptors, which are members of the low-density lipoprotein receptor (LDLR) family. Impairment in two blood-brain barrier (BBB) efflux transporters and low-density lipoprotein receptor-related protein-1 (LRP-1) are thought to contribute to the progression of Alzheimer's disease. N-aceyticysteine (Nac) has a protective effect against Aβ transporter dysfunction through an LRP-1-dependent mechanism and results in lower blood levels of interferon-γ, interleukin-3 and IL-13, in the cerebral cortex and hippocampus.

ApoE4-lipoproteins bind Aβ with lower affinity than do ApoE3-lipoproteins suggesting that ApoE4 might be less efficient in mediating Aβ clearance. In addition, ApoE might modulate Aβ removal from the brain to the systemic circulation by transporting Aβ across the blood-brain barrier. In this respect, ApoE impedes Aβ clearance at the blood-brain barrier in an isoform-specific fashion (ApoE4>ApoE3 and ApoE2), suggesting that ApoE4 inhibits Aβ clearance and/or is less efficient in mediating Aβ clearance compared with ApoE3 and ApoE2. As mentioned above, ApoE levels in CSF and plasma tend to be lower in patients with AD than in healthy individuals. Thus, increasing the expression of ApoE may prevent or slow progression of AD through acceleration of Aβ metabolism and promotion of ApoE function.

Compounds that increase ApoE expression can be considered clinically as a preventive measure. Given that expression of ApoE is controlled by peroxisome proliferator-activated receptor-γ, LXRs which act as ppar agonists are potential candidates as ApoE modulators. Indeed, recent work has demonstrated that oral administration of an LXR agonist, bexarotene, decreases Aβ plaque deposition and improves cognitive function in an ApoE-dependent manner.

Carotenoids may help prevent brain aging in an LXR agonist fashion. In one study, cognitive performance was assessed using six neuropsychological tests, and was related to dietary data obtained and measurements of baseline plasma concentrations of carotenoids (lutein, zeaxanthin, β-cryptoxanthin, lycopene, α-carotene, trans-β-carotene). A correlation between cognitive preservation and consumption of carotenoids was observed. Among the carotenoids studied, beta-cryptoxanthin and lutein exhibit LXR ligand activity and beta-cryptoxanthin was found to induce the ATP-binding cassette transporter ABCA7 mRNA

B. Comparing to a Reference Value

Once the levels of the biomarker are determined, they are compared to a reference, control or standard to determine if the subject has or is likely to develop AD, MCI, or another cognitive or neurodegenerative disorder. The reference value can be an absolute value or range of absolute values. The reference value can be a relative value or range of relative values. For example, the reference value or range of values for each biomarker can be determined by measuring the levels of the biomarker in a subject that has been previously diagnosed with AD, MCI, or another cognitive or neurodegenerative disorder (i.e., “diseased subject”). Likewise, the reference value or range of values for each biomarker can be determined by measuring the levels of the biomarker in a subject that does not have AD, MCI, or another cognitive or neurodegenerative disorder (i.e., a “normal” or “non-diseased” subject).

In some embodiments, reference values can be obtained from subjects with known scores on the mini-mental state examination (MMSE) test. The mini-mental state examination (MMSE) (also referred to as the Folstein test) is a brief 30-point questionnaire test that is used to screen for cognitive impairment and dementia. Typically, any score greater than or equal to 27 points (out of 30) indicates a normal cognition. Below this, scores can indicate severe (<9 points), moderate (10-18 points) or mild (19-24 points) cognitive impairment. The raw score may also need to be corrected for educational attainment and age. Therefore in some embodiments, subjects with a score of 26 or greater, more preferably 27 or greater are used to prepare the reference values indicative of a subject that does not have AD, MCI, or another cognitive or neurodegenerative disorder. Likewise, subjects with a score of 24 or lower can be used to prepare the reference values indicative of a subject that does have AD, MCI, or another cognitive or neurodegenerative disorder. In some embodiments, a series of reference values are prepared that can be used to establish the level of cognitive impairment of the subject (e.g., severe impairment (<9 points), moderate impairment (10-18 points) or mild impairment (19-24 points)).

In some embodiments, the reference values are established from subjects that have been diagnosed with AD, MCI, or another cognitive or neurodegenerative disease based on a measured level of a CSF biomarker. For example, in some embodiments, disease subjects used to establish reference values are those with CSF Aβ42<193 pg/mL, t-Tau>91 pg/mL, and/or t-Tau/Aβ42 ratio>0.39.

The values or range of values for controls can be determined using any suitable method known in the art, such as those discussed in more detail below.

The Examples below, and particularly Table 2, show that levels of ApoE, CRP, E-selectin, and serum amyloid protein are decreased in subjects with AD and MCI, while BNP, Cortisol, FAS, IL-3, IL-10, IL-12p40, IL-13, IL-15, Osteopontin, Pancreatic polypeptide, Resistin, and Stem cell factor are increased in subjects with MCI. Therefore, in some embodiments, the subject is determined to have, or be likely to develop AD, MCI, or another cognitive or neurodegenerative disease if the measured levels of 1, 2, 3, or all 4 of ApoE, CRP, E-selectin, or serum amyloid protein are decreased in test subjects compared to normal, non-diseased reference values. Likewise, in some embodiments, the subject is determined to have, or be likely to develop AD, MCI, or another cognitive or neurodegenerative disease if the measured levels of 1, 2, 3, 4, 5, 6, 7, 8, 9, 10, 11, 12, or 13 of ApoE, CRP, E-selectin, or serum amyloid protein are increased in test subjects compared to normal, non-diseased reference values.

In some embodiments, the comparison of the measured value and the reference value includes calculating a fold difference between the measured value and the reference value.

In particular embodiments, the methods include comparing measured values from blood samples of ApoE; BNP; CRP; pancreatic polypeptide; Cortisol; E-selectin; FAS; Gamma-IFN-induced monokine; IL-3; IL-10; IL-12p40; IL-13; IL-15; Osteopontin; Resistin; Serum amyloid protein; and Stem cell factor with reference values for samples from individuals with MMSE scores from 25 to 30, wherein this reference level is established from individuals with normal cognition. In additional examples, the method includes comparing measured values from blood samples for BNP, Cortisol, FAS, IL-3, IL-10, IL-12p40, IL-13, IL-15, Osteopontin, Pancreatic polypeptide, Resistin, Stem cell factor, ApoE, CRP, E-selectin, and serum amyloid protein with reference values for BNP, Cortisol, FAS, IL-3, IL-10, IL-12p40, IL-13, IL-15, Osteopontin, Pancreatic polypeptide, Resistin, Stem cell factor, ApoE, CRP, E-selectin, and serum amyloid protein, wherein measured values are from individuals with an MMSE score less than 25, wherein the measured value for ApoE, CRP, E-selectin, and serum amyloid protein decreases, wherein measured values for BNP, Cortisol, FAS, IL-3, IL-10, IL-12p40, IL-13, IL-15, Osteopontin, Pancreatic polypeptide, Resistin, Stem cell factor increase indicating cognitive impairment such as MCI or AD.

In another example, the method includes comparing measured values of ApoE, BNP, CRP, and Pancreatic polypeptide from blood samples with reference values of ApoE, BNP, CRP, and Pancreatic polypeptide, wherein the measured value of BNP and Pancreatic polypeptide increase, wherein the measured value of ApoE and CRP decrease, wherein CSF findings predictive of underlying AD and MCI pathologic changes (CSF Aβ42 levels<193 pg/ml and CSF t-tau/Aβ42 ratio>0.39) indicating a MCI or AD diagnosis. In one aspect, the disclosure provides methods of aiding in the diagnosis of AD or MCI by obtaining a measured level of at least one protein biomarker in a blood sample from an individual, where the protein is brain natriuretic peptide (BNP).

In a further aspect, the disclosure provides methods for monitoring progression of AD or MCI by obtaining a measured value for ApoE, BNP, CRP, and pancreatic polypeptide in blood sample; and comparing said measure value of ApoE, BNP, CRP, and pancreatic polypeptide with a reference value; wherein the measured level of BNP and Pancreatic polypeptide increases, wherein the measured levels of ApoE and CRP decrease suggests progression of MCI or AD. In certain embodiments, the measured value is obtained by measuring the level of ApoE, BNP, CRP, and pancreatic polypeptide in the blood sample.

In yet another embodiment, the disclosure provides methods of identifying candidate agents for treatment of AD and MCI by assaying a prospective candidate agent for activity in regulating the protein biomarkers, where the protein biomarker is from a group consisting of BNP, Cortisol, FAS, IL-3, IL-10, IL-12p40, IL-13, IL-15, Osteopontin, Pancreatic polypeptide, Resistin, Stem cell factor, ApoE, CRP, E-selectin, and serum amyloid protein with reference values for BNP, Cortisol, FAS, IL-3, IL-10, IL-12p40, IL-13, IL-15, Osteopontin, Pancreatic polypeptide, Resistin, Stem cell factor, ApoE, CRP, E-selectin, and serum amyloid protein. Provided herein are methods of identifying a candidate agent for treatment of MCI and AD including: assaying a prospective candidate agent for activity in the regulation of protein biomarkers, wherein said protein biomarkers are chosen from the group consisting of BNP, Cortisol, FAS, IL-3, IL-10, IL-12p40, IL-13, IL-15, Osteopontin, Pancreatic polypeptide, Resistin, Stem cell factor, ApoE, CRP, E-selectin, and serum amyloid protein with reference values for BNP, Cortisol, FAS, IL-3, IL-10, IL-12p40, IL-13, IL-15, Osteopontin, Pancreatic polypeptide, Resistin, Stem cell factor, ApoE, CRP, E-selectin, and serum amyloid protein.

Additionally, provided herein are sets of reference values for protein biomarkers including BNP, Cortisol, FAS, IL-3, IL-10, IL-12p40, IL-13, IL-15, Osteopontin, Pancreatic polypeptide, Resistin, Stem cell factor, ApoE, CRP, E-selectin, and serum amyloid protein with reference values for BNP, Cortisol, FAS, IL-3, IL-10, IL-12p40, IL-13, IL-15, Osteopontin, Pancreatic polypeptide, Resistin, Stem cell factor, ApoE, CRP, E-selectin, and serum amyloid protein and set of reagents specific for protein biomarkers, wherein said includes BNP, Cortisol, FAS, IL-3, IL-10, IL-12p40, IL-13, IL-15, Osteopontin, Pancreatic polypeptide, Resistin, Stem cell factor, ApoE, CRP, E-selectin, and serum amyloid protein with reference values for BNP, Cortisol, FAS, IL-3, IL-10, IL-12p40, IL-13, IL-15, Osteopontin, Pancreatic polypeptide, Resistin, Stem cell factor, ApoE, CRP, E-selectin, and serum amyloid protein. Specific reference values and the methods used to obtain the values are described in the Examples below.

In certain embodiments, the disclosure relates to a method including the steps of screening for plasma AD biomarkers that are used to identify a subgroup of subject for further evaluation, and confirmatory AD testing (e.g. CSF, cerebral amyloid imaging). This will lead to more efficient and cost-effective screening of subjects at high risk of AD.

Provided herein are methods for obtaining values for the comparison of the measured level to the reference level of the whole blood samples. The present disclosure provides computer readable formats including the values obtained by the methods described herein.

### IV. Devices for Detection and Data Analysis

A. Devices for Detection

In certain embodiments, the experimental blood based method of identifying biochemical biomarkers utilizes an analytical platform. In certain embodiments, the disclosure contemplates a solid surface array including probes to biomarkers disclosed herein for the purpose of detecting the biomarkers. Provided herein are devices for detection of biomarkers with surfaces including attached thereto, at least one reagent specific for each protein biomarkers in a set of proteins, wherein said set of protein biomarkers includes BNP, Cortisol, FAS, IL-3, IL-10, IL-12p40, IL-13, IL-15, Osteopontin, Pancreatic polypeptide, Resistin, Stem cell factor, ApoE, CRP, E-selectin, and serum amyloid protein with reference values for BNP, Cortisol, FAS, IL-3, IL-10, IL-12p40, IL-13, IL-15, Osteopontin, Pancreatic polypeptide, Resistin, Stem cell factor, ApoE, CRP, E-selectin, and serum amyloid protein; and at least one reagent specific for a biomarker that measures sample characteristics. In further examples, provided herein are surfaces wherein said reagent specific for said protein biomarker is an antibody, or fragment thereof, that is specific for said protein biomarker. Provided herein are combinations, including the surfaces as described herein having attached thereto at least one reagent specific for each protein biomarker and a whole blood sample from an individual. In some examples, the individual is at least 50, 60, or 65 years old.

One contemplated test setup is an immune assay, a radioimmunoassay, or a ligand binding assay, e.g., enzyme-linked immunosorbent assay. The protein biomarker in the blood sample is immobilized on a solid support such as a polystyrene microtiter plate either non-specifically by adsorption to spots or zones on the surface or specifically by capture by a ligand—molecule that has affinity for the biomarker, e.g., antibody specific to the protein biomarker. After the biomarker is immobilized presence of the marker is detected. In one example, a detection antibody (e.g., second antibody) is mixed with the surface. If the biomarker is in the spot, the detection antibody may form a complex with the biomarker. The detection antibody may be covalently linked to an enzyme that creates a signal upon exposure to appropriate conditions, e.g., by adding an enzymatic substrate to produce a visible signal which indicates the quantity of antigen in the sample. The detection antibody may be itself detected or monitored by a variety of techniques, such as through an antibody with affinity for the detection antibody conjugated to an enzyme. Typically the surface is washed to remove any proteins or antibodies that are not specifically bound.

In certain embodiments, the protein biomarker can be immobilized on the surface by ligand binding and a detection reagent will bind specifically to the biomarker. The detection reagent may be conjugated to an enzyme to generate a signal that can be quantified. For example, Rica & Stevens report an enzyme label that controls the growth of gold nanoparticles and generates colored solutions with distinct tonality when the analyte is present. See Nature Nanotechnology, 2012, 7:821-824.

In certain embodiments, the protein biomarker is captured with a ligand or antibody on a surface and the protein biomarker is labeled with an enzyme. In one example, a detection antibody conjugated to biotin or streptavidin—to create a biotin-streptavidin linkage to on an enzyme that contains biotin or streptavidin. A signal is generated by the conversion of the enzyme substrate into a colored molecule and the intensity of the color of the solution is quantified by measuring the absorbance with a light sensor. Contemplated assays may utilize chromogenic reporters and substrates that produce some kind of observable color change to indicate the presence of the protein biomarker. Fluorogenic, electrochemiluminescent, and real-time PCR reporters are also contemplated to create quantifiable signals.

Flow cytometry is a laser based technique that may be employed in counting, sorting, and detecting protein biomarkers by suspending particles in a stream of fluid and passing them by an electronic detection apparatus. A flow cytometer has the ability to discriminate different particles on the basis of color. Differential dyeing of particles with different dyes, emitting in two or more different wavelengths allows the particle to be distinguished. Multiplexed analysis allows one to perform multiple discrete assays in a single tube with the same sample at the same time.

In one example, this surface may be beads each with distinctive combinations of fluorophores that confer each bead a specified, unique color code. Beads act as a solid surface that is coated with capture antibodies of interest. Using the aliquots of the blood sample obtained, sandwich ELISA assay is then performed to detect proteins using reporter-conjugate. Ideally, this assay should be performed in duplicates, to ensure protein biomarkers identified are reproducibly found to be associated with AD or MCI. The beads are passed through a flow cell, on a laser instrument that utilizes two-laser system, in which one laser detects the color code of each bead, and the second laser detects the reporter signal, hence protein concentration. Measured values are statistically analyzed extensively to determine if an alteration in levels is associated with the clinical diagnosis of MCI or AD. If diagnostic protein biomarkers are found to be associated with either of these neurodegenerative diseases, future steps would require the development and implementation of blood based screening of the identified protein biomarkers in clinical laboratories. See Jager et al., Clin Vaccine Immunol, 2003, 10 (1) 133-139.

In certain embodiments, the particles may be polystyrene microspheres that bear carboxylate functional groups on the surface. The particles can be covalently coupled to amine-containing ligands or antibodies to a protein biomarker through surface carboxylate groups; alternatively, avidin-coupled particles can be used for binding biotinylated ligands or antibodies. The bound protein biomarker can be exposed to fluorescent antibodies or nucleic acid detection reagents to provide a specific signal for each reaction in a multiplexed assay. Each fluorescent detection reagent binds specifically to a protein biomarker that is present on only one bead set in a multiplexed assay. Fluorescent molecules may be labeled with a green-emitting fluorophore such as Bodipy® (Molecular Probes) or fluorescein isothiocyanate.

In certain embodiments, the disclosure contemplates individual sets of particles of fluorescently coded particles conjugated with ligands or antibody to protein biomarkers. After mixing the particles with a blood sample, the particles are mixed with fluorescent detection antibodies or any fluorescent molecule that will bind to the biomarkers. Mixtures of particles containing various amounts of fluorescence on their surfaces are analyzed with a flow cytometer. Data acquisition, analysis, and reporting are performed on the particles sets. As each particle is analyzed by the flow cytometer, the particle is classified into its distinct set on the basis fluorescence and values are recorded. As particles are passed through a flow cell, an instrument utilizes two-laser system wherein one laser detects the color code of each particle, and the second laser detects the reporter signal, hence protein biomarker concentration.

In some specific embodiments, the biomarker level(s) are measured using Luminex xMAP technology. Luminex xMAP is frequently compared to the traditional ELISA technique, which is limited by its ability to measure only a single analyte. The differences between ELISA and Luminex xMAP technology center mainly on the capture antibody support. Unlike with traditional ELISA, Luminex xMAP capture antibodies are covalently attached to a bead surface, effectively allowing for a greater surface area as well as a matrix or free solution/liquid environment to react with the analytes. The suspended beads allow for assay flexibility in a singleplex or multiplex format.

Commercially available formats that include Luminex xMAP technology includes, for example, BIO-PLEX® multiplex immunoassay system which permits the multiplexing of up to 100 different assays within a single sample. This technique involves 100 distinctly colored bead sets created by the use of two fluorescent dyes at distinct ratios. These beads can be further conjugated with a reagent specific to a particular bioassay. The reagents may include antigens, antibodies, oligonucleotides, enzyme substrates, or receptors. The technology enables multiplex immunoassays in which one antibody to a specific analyte is attached to a set of beads with the same color, and the second antibody to the analyte is attached to a fluorescent reporter dye label. The use of different colored beads enables the simultaneous multiplex detection of many other analytes in the same sample. A dual detection flow cytometer can be used to sort out the different assays by bead colors in one channel and determine the analyte concentration by measuring the reporter dye fluorescence in another channel.

In some specific embodiments, the biomarker(s) levels are measured using Quanterix's SIMOA™ technology. SIMOA™ technology (named for single molecule array) is based upon the isolation of individual immunocomplexes on paramagnetic beads using standard ELISA reagents. The main difference between Simoa and conventional immunoassays lies in the ability to trap single molecules in femtoliter-sized wells, allowing for a “digital” readout of each individual bead to determine if it is bound to the target analyte or not. The digital nature of the technique allows an average of 1000× sensitivity increase over conventional assays with CVs<10%. Commercially available SIMOA™ technology platforms offers multiplexing options up to a 10-plex on a variety of analyte panels, and assays can be automated.

Multiplexing experiments can generate large amounts of data. Therefore, in some embodiments, a computer system is utilized to automate and control data collection settings, organization, and interpretation.

B. Data Analysis

There are a number of statistical tests for identifying biomarkers which vary significantly between the subsets, including the conventional t test. However, as the number of biomarkers measured increases, it is generally advantageous to use a more sophisticated technique, such as SAM (see Tusher et al., 2001, Proc. Natl. Acad. Sci. U.S.A. 98(9):5116-21). Other useful techniques include Tree Harvesting (Hastie et al., Genome Biology 2001, 2:research0003.1-0003.12), Self Organizing Maps (Kohonen, 1982b, Biological Cybernetics 43(1):59-69), Frequent Item Set (Agrawal et al., 1993 “Mining association rules between sets of items in large databases.” In Proc. of the ACM SIGMOD Conference on Management of Data, pages 207-216, Washington, D.C., May 1993), Bayesian networks (Gottardo, Statistical analysis of microarray data, A Bayesian approach. Biostatistics (2001), 1,1, pp 1-37), and the commercially available software packages CART and MARS.

The SAM technique assigns a score to each biomarker on the basis of change in expression relative to the standard deviation of repeated measurements. For biomarkers with scores greater than an adjustable threshold, the algorithm uses permutations of the repeated measurements to estimate the probability that a particular biomarker has been identified by chance (calculated as a “q-value”), or a false positive rate which is used to measure accuracy. The SAM technique can be carried out using publicly available software called Significance Analysis of Microarrays (see www-stat class.stanford.edu/.about.tibs/clickwrap/sam.html).

A biomarker can be considered “identified” as being useful for aiding in the diagnosis, diagnosis, stratification, monitoring, and/or prediction of neurological disease when it is significantly different between the subsets of peripheral biological samples tested. Levels of a biomarker are “significantly different” when the probability that the particular biomarker has been identified by chance is less than a predetermined value. The method of calculating such probability will depend on the exact method utilizes to compare the levels between the subsets (e.g., if SAM is used, the q-value will give the probability of misidentification, and the p value will give the probability if the t test (or similar statistical analysis) is used). As will be understood by those in the art, the predetermined value will vary depending on the number of biomarkers measured per sample and the number of samples utilized. Accordingly, predetermined value may range from as high as 50% to as low as 20, 10, 5, 3, 2, or 1%.

As described herein, the level of at least one protein biomarker is measured in a biological sample from an individual. The protein biomarker level(s) may be measured using any available measurement technology that is capable of specifically determining the level of the biomarker in a biological sample. The measurement may be either quantitative or qualitative, so long as the measurement is capable of indicating whether the level of the biomarker in the peripheral biological fluid sample is above or below the reference value.

Although some assay formats will allow testing of peripheral biological fluid samples without prior processing of the sample, it is expected that most peripheral biological fluid samples will be processed prior to testing. Processing generally takes the form of elimination of cells (nucleated and non-nucleated), such as erythrocytes, leukocytes, and platelets in blood samples, and may also include the elimination of certain proteins, such as certain clotting cascade proteins from blood. In some examples, the peripheral biological fluid sample is collected in a container including EDTA.

The process of comparing a measured value and a reference value can be carried out in any convenient manner appropriate to the type of measured value and reference value for the biomarker at issue. As discussed above, ‘measuring’ can be performed using quantitative or qualitative measurement techniques, and the mode of comparing a measured value and a reference value can vary depending on the measurement technology employed. For example, when a qualitative colorimetric assay is used to measure biomarker levels, the levels may be compared by visually comparing the intensity of the colored reaction product, or by comparing data from densitometric or spectrometric measurements of the colored reaction product (e.g., comparing numerical data or graphical data, such as bar charts, derived from the measuring device). However, it is expected that the measured values used in the methods of the disclosure will most commonly be quantitative values (e.g., quantitative measurements of concentration, such as nanograms of biomarker per milliliter of sample, or absolute amount). As with qualitative measurements, the comparison can be made by inspecting the numerical data, by inspecting representations of the data (e.g., inspecting graphical representations such as bar or line graphs).

A measured value is generally considered to be substantially equal to or greater than a reference value if it is at least 95% of the value of the reference value (e.g., a measured value of 1.71 would be considered substantially equal to a reference value of 1.80). A measured value is considered less than a reference value if the measured value is less than 95% of the reference value (e.g., a measured value of 1.7 would be considered less than a reference value of 1.80).

The process of comparing may be manual (such as visual inspection by the practitioner of the method) or it may be automated. For example, an assay device (such as a luminometer for measuring chemiluminescent signals) may include circuitry and software enabling it to compare a measured value with a reference value for a biomarker. Alternately, a separate device (e.g., a digital computer) may be used to compare the measured value(s) and the reference value(s). Automated devices for comparison may include stored reference values for the biomarker(s) being measured, or they may compare the measured value(s) with reference values that are derived from contemporaneously measured reference samples.

In some embodiments, the methods of the disclosure utilize ‘simple’ or ‘binary’ comparison between the measured level(s) and the reference level(s) (e.g., the comparison between a measured level and a reference level determines whether the measured level is higher or lower than the reference level). For protein biomarkers, a comparison showing that the measured value for the biomarker is lower than the reference value indicates or suggests a diagnosis of AD or MCI.

As described herein, biological fluid samples may be measured quantitatively (absolute values) or qualitatively (relative values). The respective biomarker levels for a given assessment may or may not overlap. As described herein, for some embodiments, qualitative data indicate a given level of cognitive impairment (mild, moderate or severe AD) (which can be measured by MMSE scores) and in other embodiments, quantitative data indicate a given level of cognitive impairment.

In certain aspects of the disclosure, the comparison is performed to determine the magnitude of the difference between the measured and reference values (e.g., comparing the ‘fold’ or percentage difference between the measured value and the reference value). A fold difference that is about equal to or greater than the minimum fold difference disclosed herein suggests or indicates a diagnosis of AD, MCI, progression from MCI to AD, or progression from mild AD to moderate AD, as appropriate to the particular method being practiced. A fold difference can be determined by measuring the absolute concentration of a protein and comparing that to the absolute value of a reference, or a fold difference can be measured by the relative difference between a reference value and a sample value, where neither value is a measure of absolute concentration, and/or where both values are measured simultaneously. A fold difference may be in the range of 10% to 95%. An ELISA measures the absolute content or concentration of a protein from which a fold change is determined in comparison to the absolute concentration of the same protein in the reference. An antibody array measures the relative concentration from which a fold change is determined. Accordingly, the magnitude of the difference between the measured value and the reference value that suggests or indicates a particular diagnosis will depend on the particular biomarker being measured to produce the measured value and the reference value used (which in turn depends on the method being practiced).

In some embodiments, the p valve of the measured protein level compared to the control or reference value is a less than 0.1, preferably less the 0.05, more preferably less than 0.01.

In some embodiments statistical analysis, computational analysis, and/or other analytical techniques are employed to predict presence or likelihood of developing AD, MCI, or another cognitive or neurodegenerative disease, or in assessing probability of clinical outcome. Researchers currently use statistical techniques such as clustering and statistical mining to distill through large quantities of data, for example, calculating co-variances between the measurements.

For example, a computer-implemented method of analyzing a dataset can include a computer system with a network inference engine which can generate a forward simulation risk model using analytical techniques known to those skilled in the art, including but not limited to metropolis Monte Carlo methods, a Bayesian scoring method, Bayesian Belief propagator, etc. Other data-driven techniques may include computational representations of the causal relationships between independent variables which include, but are not limited to one or more of the biomarkers disclosed herein and optionally, DNA alterations, changes in mRNA, protein, metabolites, phenotypes and electronic medical records which utilize a probabilistic modeling framework to assess risk of AD, presence of AD or response to a specific treatment.

The method of analysis can be used in concert with other bioinformatics tools known to one of skill in the art. It should be appreciated that diverse data types (e.g., molecular, phenotypic, etc.) may increase the degree of robustness of the predictive model, and while analyses disclosed herein typically include blood protein biomarker analysis, any other relevant data can also be included to refine the risk stratification and/or predictive response to therapy based upon said data. For example, the systems and methods can be used to appropriately segregate patient population into appropriate training groups, make predictions of prognosis and or therapeutic response, predict disease phenotype and or therapeutic response or used to help in clinical trial design and optimization. The systems and methods can also be used to identify drug targets for Alzheimer's therapies on a patient-specific basis, used to validate biomarkers and targets for therapeutics from clinical data from patients, all of which can be included in the computational method to arrive at a digitally displayed risk score or response to treatment.

The systems and methods described herein include methods for selecting one or more treatments for a patient given certain patient-specific input conditions, observing whether said patient-specific conditions result in a prediction that the one or more therapeutics will be effective in said patients, reporting said prediction to said clinicians, the design of clinical trials, and other relevant clinical uses related to the care of individuals at risk for AD, MCI, or another cognitive or neurodegenerative disease.

The methods can include regressing the theoretical estimated distribution of protein concentrations against observed values, identifying outlier data points as data points having significant influence in the estimation of the parameters of the log-normal distribution, removing the outlier data points from the dataset, recalculating the parameters of the distribution, and replacing the outlier data points with the maximum likelihood estimate for the distribution. In certain embodiments, the dataset includes data from two or more patients and contains differences between said two or more patients. In such embodiments, the differences exist with respect to one or more of the biomarkers disclosed herein and optionally one or more of the following: genes, regions of DNA, RNA, miRNA, proteins, modified proteins, and clinical endpoints. Data for analysis can include gene or gene expression microarrays, proteomics, metabolomics electronic medical records (EMR) and updated patient specific data, and the like. In addition to the molecular profiling data, clinical response measurements, and clinical features of a population of patients can be included in this analysis. Example 4 describes exemplary methods for analyzing the data.

### V. Kits

The disclosure provides kits for carrying out any of the methods described herein. Kits of the disclosure may comprise at least one reagent specific for a protien biomarker, and may further include instructions for carrying out a method described herein. Kits may also comprise protein biomarker reference samples, that is, useful as reference values.

In certain embodiment, the disclosure provides kits for diagnosing AD and MCI including at least one reagent specific for a protein biomarker, where the protein biomarker is from the group consisting of BNP, Cortisol, FAS, IL-3, IL-10, IL-12p40, IL-13, IL-15, Osteopontin, Pancreatic polypeptide, Resistin, Stem cell factor, ApoE, CRP, E-selectin, and serum amyloid protein with reference values for BNP, Cortisol, FAS, IL-3, IL-10, IL-12p40, IL-13, IL-15, Osteopontin, Pancreatic polypeptide, Resistin, Stem cell factor, ApoE, CRP, E-selectin, and serum amyloid protein and instructions for carrying out a method of aiding in the diagnosis of MCI and AD described herein.

In yet another aspect, the disclosure provides kits for monitoring progression of AD and MCI in patients including at least one reagent specific for BNP; and instructions for carrying out a method of monitoring AD and MCI progression described herein. In a further aspect, the disclosure provides kits for stratifying AD and MCI patients including at least one reagent specific for BNP, at least one reagent specific for ApoE, at least one reagent specific for CRP, at least one reagent specific for pancreatic polypeptide, and instructions for carrying out a method of stratifying an AD and MCI patient described herein. In yet further examples kits including protein biomarkers selected from the group consisting BNP, ApoE, CRP, and pancreatic polypeptide. In further examples of kits, the reagent specific for the protein biomarkers is an antibody, or fragment thereof, that is specific for said protein biomarkers. In further examples kits further comprise at least one reagent specific for a biomarker that measures sample characteristics.

More commonly, kits of the disclosure comprise at least two different biomarker-specific affinity reagents, where each reagent is specific for a different biomarker. In some embodiments, kits comprise at least 3, at least 4, at least 5, at least 6, at least 7, at least 8, at least 9, or at least 10 reagents specific for an biomarker. In some embodiments, the reagent(s) specific for an biomarker is an affinity reagent. In certain embodiments, the reagent are specific for Apolipoprotein E (apoE); B-type natriuretic peptide; C-reactive protein; and pancreatic polypeptide. In certain embodiments, the reagent are specific for Apolipoprotein E (apoE), B-type natriuretic peptide, C-reactive protein, pancreatic polypeptide, Cortisol, E-selectin, FAS, Gamma-IFN-induced monokine, IL-3, IL-10, IL-12p40, IL-13, IL-15, Osteopontin, Resistin, Serum amyloid protein, and Stem cell factor.

Kits including a single reagent specific for an biomarker will generally have the reagent enclosed in a container (e.g., a vial, ampoule, or other suitable storage container), although kits including the reagent bound to a substrate (e.g., an inner surface of an assay reaction vessel) are also contemplated. Likewise, kits including more than one reagent may also have the reagents in containers (separately or in a mixture) or may have the reagents bound to a substrate.

In some embodiments, the biomarker-specific reagent(s) will be labeled with a detectable marker (such as a fluorescent dye or a detectable enzyme), or be modified to facilitate detection (e.g., biotinylated to allow for detection with an avidin- or streptavidin-based detection system). In other embodiments, the biomarker-specific reagent will not be directly labeled or modified.

Certain kits of the disclosure will also include one or more agents for detection of bound biomarker-specific reagent. As will be apparent to those of skill in the art, the identity of the detection agents will depend on the type of biomarker-specific reagent(s) included in the kit, and the intended detection system. Detection agents include antibodies specific for the biomarker-specific reagent (e.g., secondary antibodies), primers for amplification of an biomarker-specific reagent that is nucleotide based (e.g., aptamer) or of a nucleotide ‘tag’ attached to the biomarker-specific reagent, avidin- or streptavidin-conjugates for detection of biotin-modified biomarker-specific reagent(s), and the like.

A modified substrate or other system for capture of biomarkers may also be included in the kits of the disclosure, particularly when the kit is designed for use in a sandwich-format assay. The capture system may be any capture system useful in a biomarker assay system, such as a multi-well plate coated with a biomarker-specific reagent, beads coated with an biomarker-specific reagent, and the like.

In certain embodiments, kits according to the disclosure include the reagents in the form of an array. The array includes at least two different reagents specific for biomarkers (each reagent specific for a different Biomarker) bound to a substrate in a predetermined pattern (e.g., a grid). Accordingly, the present disclosure provides arrays including Apolipoprotein E (apoE), B-type natriuretic peptide, C-reactive protein, and pancreatic polypeptide. In certain embodiments, the present disclosure provides arrays including Apolipoprotein E (apoE), B-type natriuretic peptide, C-reactive protein, pancreatic polypeptide, Cortisol, E-selectin, FAS, Gamma-IFN-induced monokine, IL-3, IL-10, IL-12p40, IL-13, IL-15, Osteopontin, Resistin, Serum amyloid protein, and Stem cell factor.

The instructions relating to the use of the kit for carrying out the disclosure generally describe how the contents of the kit are used to carry out the methods of the disclosure. Instructions may include information as sample requirements (e.g., form, pre-assay processing, and size), steps necessary to measure the Biomarker(s), and interpretation of results.

Instructions supplied in the kits of the disclosure are typically written instructions on a label or package insert (e.g., a paper sheet included in the kit), but machine-readable instructions (e.g., instructions carried on a magnetic or optical storage disk) are also acceptable. In certain embodiments, machine-readable instructions comprise software for a programmable digital computer for comparing the measured values obtained using the reagents included in the kit.

### VI. Systems for Measuring Protein Levels

In certain embodiments, the methods may be implemented by computers, systems, or stored on a computer-readable storage medium as instructions for detecting the protein biomarkers.

In some embodiments, the disclosure relates to a system. The system may include a computer having a processor configured to perform the methods of the disclosure. The system may also include or may communicate with a fluorescent camera or other device that can measure light or a change in current of an electrode or system configured to subject a sample to testing device.

In some embodiments, the system may include a computer having a processor configured to perform the methods of the disclosure. In certain embodiments, the method contemplates recording measurements and/or diagnosis and/or second line chemotherapy treatment on a computer readable medium as data. In certain embodiments the disclosure, contemplates reporting measurements or diagnosis to the subject, a medical professional, or a representative thereof. In certain embodiments, the disclosure contemplates transferring recorded data over the internet from a diagnostic lab to a computer in a medical facility.

In some embodiments, the disclosure relates to a system for measuring and recording the protein biomarkers disclosed herein including a visual device with a probe that binds to the biomarkers and computer readable memory.

In some embodiments, the method further includes outputting quantification results. In some embodiments, the method may further comprise recording the detected changes on a computer-readable medium through a visual device such as a camera or video recorder. In certain embodiments, the disclosure contemplates calculating fluorescent intensity and correlating it to a reference sample with a known quantity of the biomarker.

In some embodiments, the measuring protein levels may be outputted from a visual device through fluorescence. In some embodiments, the outputting may include displaying, printing, storing, and/or transmitting the measured fluorescence or protein levels. In some embodiments, the measured fluorescence or protein levels may be transmitted to another system, server and/or storage device for the printing, displaying and/or storing.

The methods of the disclosure are not limited to the steps described herein. The steps may be individually modified or omitted, as well as additional steps may be added.

Unless stated otherwise as apparent from the following discussion, it will be appreciated that terms such as “detecting,” “receiving,” “quantifying,” “mapping,” “generating,” “registering,” “determining,” “obtaining,” “processing,” “computing,” “deriving,” “estimating,” “calculating” “inferring” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods may be compiled for execution on a variety of hardware platforms and for interface to a variety of operating systems. In addition, embodiments are not described with reference to any particular programming language. It will be appreciated that a variety of programming languages may be used to implement embodiments of the disclosure.

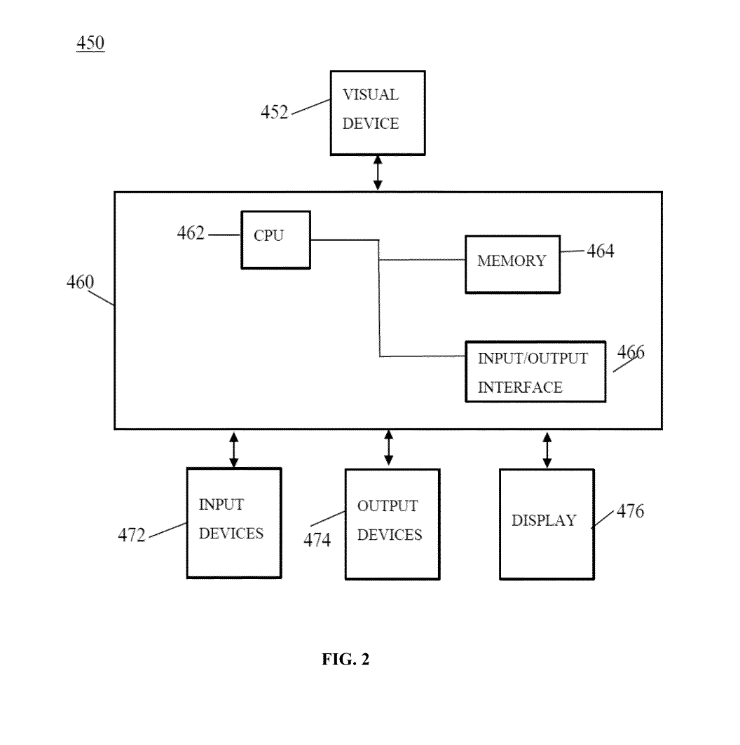

FIG. 2 shows an example of a system 450 that may be used to quantify measured fluorescence or protein levels detected by the sensor according to embodiments. The system 450 may include any number of modules that communicate with others through electrical or data connections. In some embodiments, the modules may be connected via a wired network, wireless network, or combination thereof. In some embodiments, the networks may be encrypted. In some embodiments, the wired network may be, but is not limited to, a local area network, such as Ethernet, or wide area network. In some embodiments, the wireless network may be, but is not limited to, any one of a wireless wide area network, a wireless local area network, a Bluetooth network, a radio frequency network, or another similarly functioning wireless network.

Although the modules of the system are shown as being directly connected, the modules may be indirectly connected to one or more of the other modules of the system. In some embodiments, a module may be only directly connected to one or more of the other modules of the system.

It is also to be understood that the system may omit any of the modules illustrated and/or may include additional modules not shown. It is also be understood that more than one module may be part of the system although one of each module is illustrated in the system. It is further to be understood that each of the plurality of modules may be different or may be the same. It is also to be understood that the modules may omit any of the components illustrated and/or may include additional component(s) not shown.

In some embodiments, the modules provided within the system may be time synchronized. In further embodiments, the system may be time synchronized with other systems, such as those systems that may be on the medical and/or research facility network.

The system 450 may optionally include a visual device 452. The visual device 452 may be any visual device configured to capture changes in a shape, light, or fluorescence. For example, the visual device may include but is not limited to a camera and/or a video recorder. In some embodiments, the visual device may be a part of a microscope system. In certain embodiments, the system 450 may communicate with other visual device(s) and/or data storage device.

In some embodiments, the visual device 552 may include a computer system to carry out the image processing. The computer system may further be used to control the operation of the system or a separate system may be included.

The system 450 may include a computing system 460 capable of quantifying the expression. In some embodiments, the computing system 460 may be a separate device. In other embodiments, the computing system 460 may be a part (e.g., stored on the memory) of other modules, for example, the visual device 452, and controlled by its respective CPUs.

The system 460 may be a computing system, such as a workstation, computer, or the like. The system 460 may include one or more processors (CPU) 462. The processor 462 may be one or more of any central processing units, including but not limited to a processor, or a microprocessor. The processor 462 may be coupled directly or indirectly to one or more computer-readable storage medium (e.g., physical memory) 464. The memory 464 may include one or more memory elements, such random access memory (RAM), read only memory (ROM), disk drive, tape drive, etc., or a combinations thereof. The memory 464 may also include a frame buffer for storing image data arrays. The memory 464 may be encoded or embedded with computer-readable instructions, which, when executed by one or more processors 462 cause the system 460 to carry out various functions.

In some embodiments, the system 460 may include an input/output interface 468 configured for receiving information from one or more input devices 472 (e.g., a keyboard, a mouse, joystick, touch activated screen, etc.) and/or conveying information to one or more output devices 474 (e.g., a printing device, a CD writer, a DVD writer, portable flash memory, display 476 etc.). In addition, various other peripheral devices may be connected to the computer platform such as other I/O (input/output) devices.

In some embodiments, the disclosed methods may be implemented using software applications that are stored in a memory and executed by a processor (e.g., CPU) provided on the system. In some embodiments, the disclosed methods may be implanted using software applications that are stored in memories and executed by CPUs distributed across the system. As such, the modules of the system may be a general purpose computer system that becomes a specific purpose computer system when executing the routine of the disclosure. The modules of the system may also include an operating system and micro instruction code. The various processes and functions described herein may either be part of the micro instruction code or part of the application program or routine (or combination thereof) that is executed via the operating system.

It is to be understood that the embodiments of the disclosure may be implemented in various forms of hardware, software, firmware, special purpose processes, or a combination thereof. In one embodiment, the disclosure may be implemented in software as an application program tangible embodied on a computer readable program storage device. The application program may be uploaded to, and executed by, a machine including any suitable architecture. The system and/or method of the disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

It is to be further understood that, because some of the constituent system components and method steps depicted in the accompanying figures may be implemented in software, the actual connections between the systems components (or the process steps) may differ depending upon the manner in which the disclosure is programmed. Given the teachings of the disclosure provided herein, one of ordinary skill in the related art will be able to contemplate these and similar implementations or configurations of the disclosure.

### VII. Methods of Identifying Biomarkers for AD

Methods for identifying AD protein biomarkers useful for assisting in the diagnosis, monitoring the progression, or determining an individual's risk for MCI or AD or other neurodegenerative diseases are also provided. The diagnostic protein biomarkers measured in the practice of the embodiment may be one or more proteinaceous marker described above.

In some embodiments, the methods of the disclosure are carried out by obtaining a set of measured values for a panel of protein biomarkers from of blood samples and comparing them to control values, for example, a value indicative of a disease or healthy state, and determining that the biomarker is correlated with a diseased or healthy state.

The process of comparing the measured values may be carried out by any method known in the art, including SPSS 17.0, Significance Analysis of Microarrays (SAM), PASS 11, Intersection Union Test (IUT), and Bonferroni correction.

In one aspect, the disclosure provides methods for identifying one or more protein biomarkers helpful for the diagnosis of AD or MCI, by obtaining measured values from a set of blood samples for a panel of protein biomarkers, wherein the set of blood samples is divisible into subsets on the basis of cognitive ability, comparing the measured values from each subset for at least one protein biomarker; and identifying at least one protein biomarker for which the measured values are significantly different between the subsets. In some embodiments, the comparing process is carried out using Significance Analysis of Microarrays. In certain embodiments, the neurodegenerative disease is from the group consisting of Alzheimer's disease, MCI, or very mild dementia.

In another aspect, the disclosure provides methods for identifying at least one protein biomarker helpful for assisting in the diagnosis of AD and MCI by obtaining measured values from a set of blood samples for a panel of protein biomarkers, wherein blood samples is divisible into subsets on the basis of cognitive ability, comparing the measured values from each subset for at least one protein biomarker; and identifying protein biomarkers for which the measured values are considerably different between the subsets.

The following Examples are provided to illustrate the disclosure, but are not intended to limit the scope of the disclosure in any way.

### EXAMPLES

### Example 1

### Univariate Analysis in Penn and WU Cohorts. Materials and Methods

Participates

Subjects in the 2 discovery sets were recruited and longitudinally followed at Penn and WU (Table 1), while subjects in ADNI are provided in Trojanowski et al., Alzheimers Dement, 2010, 6:230-238 and Petersen et al., Neurology, 2010, 74:201-209. At Penn, participants (n is 267) were community-dwelling healthy volunteers and patients evaluated at subspecialty clinics dedicated to the evaluation of neurodegenerative disorders including MCI, AD, and related dementia. Cognitively normal subjects were recruited through all subspecialty clinics to participate in biofluid studies. APOE genotyping was performed for 235 out of 267 Penn subjects. At WU, participants (n is 333) were community-dwelling volunteers enrolled in longitudinal studies of healthy aging and dementia at the Knight AD Research Center at Washington University. Clinical diagnosis was evaluated based on criteria from the National Institute of Neurological and Communicative Diseases and Stroke-Alzheimer's Disease and Related Disorders Association. See McKhann Neurology, 1984, 34:939-944. Cognitive status was rated with the Clinical Dementia Rating scale (CDR): CDR of 0 indicates no dementia, CDR 0.5 indicates very mild dementia, and CDR 1 indicates mild dementia. Some of the CDR 0.5 participants in the study met the criteria for MCI and some were less impaired and were considered “pre-MCI.” APOE genotyping was performed for subjects enrolled at WU.

Procedures

Samples were collected from Penn and WU subjects according to strict protocols without protease inhibitors. At sample collection, participants were about or greater than 50 years of age and in good general health (including no evidence of clinically significant liver disease or renal failure), having no other psychiatric or medical diagnoses that could contribute importantly to cognitive impairment or dementia other than the primary neurodegenerative disorder. At Penn, plasma was collected in 10 mL K2EDTA tubes (BD Vacutainer®) without overnight fasting and refrigerated immediately (4° C.) before transporting to a central site on ice for centrifuge (2,000 g at 15 minutes at 4° C.) separation into plasma and cellular components within 4 hours of collection Plasma aliquots (0.5 mL) were prepared, bar-coded, and then stored in polypropylene vials at negative 80° C. until analysis. Quality control samples to determine coefficients of variation (CV) included duplicate plasma samples from 3 control subjects analyzed at the same time as the remaining Penn subjects, and an average intra-assay CV was obtained for each analyte of interest. At WU, plasma was collected in polypropylene tubes after overnight fasting between 7:30 and 8:00 AM and centrifuged (2,000 g at 15 minutes at 4° C.) for separation into plasma and cellular components. Plasma aliquots (0.5 mL) were stored at negative 80° C. until analyzed.

Plasma aliquots from each center were interrogated consecutively in 2 batches (1 batch per center) in 2009 by Rules-Based Medicine (RBM, Austin, Tex.) for levels of 190 analytes using the multiplex Human DiscoveryMAP™ panel and a Luminex 100 platform. The 190 analytes were assembled into preformatted assays designed for different diseases including cancer, autoimmune disorders, AD, Parkinson disease, and frontotemporal degeneration. Plasma levels of 190 analytes in 566 subjects from the ADNI cohort (Table 1) were also measured at RBM in 2010 using the same multiplexed immunoassays. Analytes below threshold of detection (11 for Penn and 21 for WU) were excluded. Dynamic range for each plasma analyte in the RBM panel is provided on the ADNI Web site (http://adni.loni.ucla.edu). A total of 352 subjects (56 normal cognition, 195 MCI, and 101 AD) also had CSF AD biomarker levels provided by the ADNI Biomarker Core.

Statistical Analysis

Statistical analysis in this study was performed in SPSS 17.0 (Chicago, Ill.) and significance analysis of microarrays (SAM). See Tibshirani et al., Proc. Natl. Acad. Sci. USA 2002; 99:6567-6572. In each cohort, cognitively impaired individuals (Penn: MCI and AD, WU: CDR 0.5 and 1) were grouped together in an effort to identify plasma analytes altered across various stages of the very mild dementia/MCI/AD spectrum, and because of the differential distribution of subjects within each impaired category. Power calculation was performed in PASS 11 (Kaysville, Utah), which showed 89.0% power in the Penn cohort and 93.8% power in the WU cohort for each of 190 analytes to detect a true difference in expression of at least 0.5 with estimated group SD of 1.0 and a false discovery rate of 0.10 using a 2-sided 2-sample t test. All raw levels were log transformed to achieve normal distribution. For initial identification of individual analytes different between normal cognition and very mild dementia/MCI/AD, logistic regression model was used adjusting for age and gender.

A model based on Intersection Union Test (IUT) was used, which involves identification of overlapping results (analytes in the current study, genes in microarray studies) from distinct datasets. Quan et al., Stat. Med. 2001; 20:3159-3173. As this method may be overly conservative and reduce the power in detecting true positives, a more liberal threshold of significance was used at the univariate analysis stage of p less than 0.10 (after adjusting for age and gender) to reduce type II errors. Type I errors were reduced by applying 2 additional filters by identifying 1) analytes from the modified IUT with common direction of change (vector direction) and 2) analytes from (1) that fulfill strict Bonferroni correction at the validation phase. Analytes with similar associations with very mild dementia/MCI/AD in each discovery cohort were then analyzed in the ADNI cohort (n is 566) for association with the diagnosis of MCI/AD with an a value of 0.0036 (0.05/14) for the 14 analytes that passed first level screening. Univariate analysis was also repeated within each cohort using SAM, and analytes found to be significant in more than 2 cohorts were identified.

In addition, the relationship between plasma MAP biomarkers and CSF AD biomarker-drive diagnosis (CSF Aβ42 levels greater than 193 pg/mL and t-tau/Aβ42 less than 0.39) were determined adjusting for age and gender (p less than 0.0036), and the correlation between CSF AD biomarker and plasma marker levels using linear regression analysis. In these models, CSF AD biomarker (Aβ42, t-tau/Aβ42) levels were dependent variables, age and gender were entered in the first stage as independent variables, and number of APOE4 alleles and plasma biomarker levels were then entered in a stepwise fashion. Finally, as pancreatic peptide levels are influenced by cholinesterase inhibitor (ChEI) therapy, the correlation between plasma and CSF AD biomarkers was analyzed among subjects without ChEI (including donepezil, galantamine, and rivastigmine), including 58 subjects with normal cognition, 226 subjects with MCI, and 20 subjects with AD.

### Results

Plasma samples from subjects in two separate cohorts from Penn and WU were analyzed to identify any alterations in protein levels that correlated with AD or MCI. Table 1 lists the demographic features of subjects included in plasma multianalyte profiling from the University of Pennsylvania, Washington University, and Alzheimer's Disease Neuroimaging Initiative. a APOE genotyping information missing in 42 subjects, with total number of subjects with genotyping information shown in parentheses. The WU cohort had a higher proportion of participants with normal cognition (73% vs 55%, p less than 0.0001) and a lower proportion of subjects with clinically probable AD than the Penn cohort (8% vs 38%, p less than 0.001).

TABLE 1
Demographic Features of Subjects Included in Plasma Multianalyte Profiling

| University of Pennsylvania | Normal cognition | MCI | AD | Other dementias |
|---|---|---|---|---|
| No. (% female) | 126 (64) | 16 (94) | 88 (55) | 37 (41) |
| Age, y (SD) | 68.30 (10.87) | 72.38 (8.60) | 70.83 (11.69) | 65.14 (9.80) |
| % (n) APOE4 postitive^{a} | 22 (101) | 80 (15) | 58 (74) | 43 (35) |
| MMSE (SD) | 29.15 (1.15) | 25.19 (2.26) | 17.59 (6.70) | |

| Washington University | Normal cognition | CDR 0.5 | CDR 1 |
|---|---|---|---|
| No. (% female) | 242 (65) | 63 (52) | 28 (50) |
| Age, y (SD) | 71.6 (7.4) | 74.6 (7.3) | 76.8 (6.2) |
| % APOE4 positive | 32 | 54 | 57 |
| MMSE (SD) | 28.9 (1.3) | 26.3 (2.8) | 22.5 (4.0) |
| ADNI | Normal cognition | MCI | AD |
| No. (% female) | 58 (48.3) | 396 (35.4) | 112 (42) |
| Age, y (SD) | 75.2 (5.8) | 74.9 (7.5) | 75.0 (8.0) |
| % APOE4 positive | 9 | 53 | 68 |
| MMSE | 28.9 (1.2) | 27.0 (1.8) | 23.6 (1.9) |
| CDR 0 | 58 | 1 | 0 |
| CDR 0.5 | 0 | 395 | 59 |
| CDR 1 | 0 | 0 | 53 |
| CSF (n = 352) | | | |
| Aβ42 (SD) | 251.45 (20.47) | 163.81 (54.18) | 142.52 (39.75) |
| t-Tau (SD) | 63.69 (23.56) | 103.66 (61.00) | 121.21 (57.12) |

Out of the 190 proteins analyzed 41 proteins from the Penn group and 51 proteins from WU were found to be associated with very mild dementia, MCI, and AD (p<0.10). Out of the 23 analytes that were found in both cohorts, alipoprotein A1, alipoprotein H, cystatin C, fibrinogen, myeloperoxidase, and neutrophil, gelatinase-associated lipocalin, all showed changes in protein levels in opposite directions in the association with a diagnosis and were excluded from the analysis (Table 2).

Among the 17 proteins remaining in the study, 5 were identified in a previous publication that used the RBM panel to identify the association between analytes and clinical AD (Table 2). See O'Bryant et al., A blood-based screening tool for Alzheimer's disease that spans serum and plasma: findings from TARC and ADNI, PloS one, 2011, 6:e28092. These five analytes include C-reactive protein (CRP), interleukin (IL)-10, IL-15, pancreatic polypeptide, and resistin. IL-3 was also identified in another report using different multiplex platform to analyze plasma samples. In this study IL-3 was shown to have an opposite direction of association with AD. Significance analysis of microarrays (SAM) was used to identify analytes associated with very mild dementia, MCI, and AD in both cohorts, with 6 protein biomarkers, al-antitrypsin, ApoE, CRP, N-terminal pro B-type natriuretic peptide, osteopontin, and serum amyloid P.

TABLE 2
Identification of Biomarkers Associated with AD

| Analyte | Penn | WU |
|---|---|---|
| Apolipoprotein A1 | 1.044 | 0.972 |
| Apolipoprotein E | 0.945 | 0.965 |
| Apolipoprotein H | 1.044 | 0.977 |
| Brain natriuretic peptide | 1.083 | 1.074 |
| Cortisol | 1.065 | 1.035 |
| C-reactive protein | 0.824 | 0.921 |
| Cystatin C | 1.035 | 0.972 |
| E-selectin | 0.946 | 0.962 |
| FAS | 1.031 | 1.017 |
| Fibrinogen | 1.039 | 0.982 |
| Gamma-IFN-induced monokine | 1.066 | 0.956 |
| IL-3 | 1.054 | 1.066 |
| IL-10 | 1.039 | 1.023 |
| IL-12p40 | 1.017 | 1.065 |
| IL-13 | 1.050 | 1.045 |
| IL-15 | 1.031 | 1.046 |
| Myeloperoxidase | 1.051 | 0.880 |
| NGAL | 1.041 | 0.982 |
| Osteopontin | 1.122 | 1.033 |
| Pancreatic polypeptide | 1.078 | 1.093 |
| Resistin | 1.059 | 1.022 |
| Serum amyloid protein | 0.958 | 0.440 |
| Stem cell factor | 1.039 | 1.054 |

The first column of Table 2 provides a list of biomarkers identified in the screen, and includes the seventeen protein biomarkers that were found to be associated with AD and MCI in both the University of Pennsylvania and Washington University cohorts. The list of biomarkers is followed by a column listing the odds ratios associated with very mild dementia, MCI, and AD. Values greater than 1 indicate an increase in the log-transformed protein level is associated with AD, while values less than 1 indicate a decrease in the log-transformed protein level. Abbreviations: IFN is interferon; IL is interleukin.

### Example 2

### Univariate Analysis in ADNI Cohort

The association of the seventeen protein biomarkers with a clinical diagnosis of AD or MCI was confirmed in a third cohort from Alzheimer's Disease Neuroimaging Institute (ADNI). In the ADNI group, B-type natriuretic peptide (BNP) levels were examined instead of N-terminal pro B-type natriuretic peptide levels, and IL-10, IL-12p40, and IL-15 levels were not available. Using Bonferroni correction for the 14 protein markers (p<0.0036), six analytes were found to be highly associated with clinical diagnosis of MCI or AD. The analytes identified including ApoE, BNP, cortisol, CRP, IL-3, and pancreatic polypeptide. 352 participants from the ADNI cohort with CSF were analyzed to determine if CSF findings that are predictive of the underlying AD pathology (i.e., CSF Aβ42 levels<193 pg/mL and CSF t-tau/Aβ42 ratio<0.39) correlated with the altered levels of protein biomarkers identified. This analysis found four plasma proteins, including apoE, BNP, CRP, and pancreatic polypeptide were highly associated with established CSF AD biomarker levels, with all analytes having acceptable intra-assay variability. Using SAM with a false discovery rate of 10%, and then IUT to all 3 datasets yielded 14 protein biomarkers associated with mild dementia, MCI, and AD including apoE, CRP, and insulin growth factor binding protein 2 (IGF-BP2) being altered in all 3 cohorts, and BNP and pancreatic peptide altered in 2 cohorts. Given the larger type II error associated with applying IUT to all 3 datasets, these results were considered to be consistent with the discovery-validation approach using logistic regression.

TABLE 3
Effects of Clinical or CSF-Based Diagnosis on Analyte Levels

| | |
|---|---|
| Odds ratio | p Value |

Diagnosis of MCI or AD

| | | |
|---|---|---|
| ApoE^{b} | 0.881 | <0.001 |
| BNP^{b} | 1.230 | <0.001 |
| CRP^{b} | 0.824 | <0.001 |
| IL-3^{b} | 1.141 | <0.001 |
| PP^{b} | 1.171 | <0.001 |
| Cortisol | 1.030 | 0.015 |
| E-selectin | 0.636 | 0.134 |
| FAS | 1.017 | 0.19 |
| IGF-BP2 | 0.984 | 0.419 |
| IL-13 | 1.029 | 0.097 |
| Osteopontin | 0.947 | 0.006 |
| Resistin | 1.004 | 0.822 |
| SAP | 0.987 | 0.287 |
| SCF | 1.036 | 0.063 |

CSF Aβ42 < 193 pg/mL

| | | |
|---|---|---|
| ApoE | 0.901 | <0.001 |
| BNP | 1.175 | <0.001 |
| CRP | 0.798 | <0.001 |
| PP | 1.129 | <0.001 |
| Cortisol | 1.031 | 0.004 |
| E-selectin | 0.673 | 0.098 |
| FAS | 1.001 | 0.929 |
| IGF-BP2 | 1.003 | 0.873 |
| IL-3 | 1.050 | 0.055 |
| IL-13 | 1.005 | 0.735 |
| Osteopontin | 0.973 | 0.118 |
| Resistin | 1.006 | 0.647 |
| SAP | 0.993 | 0.511 |
| SCF | 0.997 | 0.858 |

CSF t-tau/Aβ42 > 0.39

| | | |
|---|---|---|
| ApoE | 0.921 | <0.001 |
| BNP | 1.113 | <0.001 |
| Cortisol | 1.033 | 0.003 |
| CRP | 0.770 | <0.001 |
| PP | 1.115 | 0.001 |
| E-selectin | 0.600 | 0.031 |
| FAS | 0.993 | 0.523 |
| IGF-BP2 | 1.035 | 0.047 |
| IL-3 | 1.035 | 0.171 |
| IL-13 | 1.000 | 0.974 |
| Osteopontin | 0.970 | 0.078 |
| Resistin | 1.001 | 0.965 |
| SAP | 0.991 | 0.373 |
| SCF | 1.003 | 0.859 |

Table 3 lists the effects of clinical or CSF-based diagnosis on analyte levels in the ADNI cohort (adjusted for age and gender). Levels of IL-10, IL-12p40, and IL-15 were not available in the ADNI cohort. ^{b} Analytes identified from both Penn and WU cohorts significantly associated with a clinical diagnosis of MCI/AD or CSF biomarker pattern associated with pathologic AD with odds ratios shown. Abbreviations: Aβ42 is amyloid 1-42; BNP is brain natriuretic peptide; CRP is C-reactive protein; IGF-BP2 is insulin-like growth factor binding protein 2; IL is interleukin; PP is pancreatic polypeptide; SAP is serum amyloid protein; SCF is stem cell factor; t-tau is total tau.

### Example 3

### Correlation Between Plasma and CSF AD Biomarkers

Altered levels of these AD biomarkers were found to be associated with an AD clinical diagnosis and AD CSF marker profiles, however, the direct association of CSF AD biomarker levels with the identified protein biomarkers remained to be elucidated. Multivariate linear regression modeling indicated that CSF Aβ42 levels were strongly correlated with number of APOE4 alleles adjusted for age and gender (p<0.001, R=0.559, adjusted R2 of 0.307), BNP levels and pancreatic polypeptide (R=0.596, R2 of 0.345). In addition, a similar correlation was found with CSF t-tau/Aβ42 ratios, however a weaker relationship was found between an increased t-Tau/Aβ42 ratio and candidate AD protein biomarkers (number of APOE4 alleles and plasma pancreatic polypeptide levels, R<0.404, adjusted R2 of 0.154). ApoE levels were found to be associated with APOE4 allele frequency and were not predictive of CSF biomarker levels independently of the latter. The addition of IGF-BP2 levels from SAM analysis or adjustment for ChEI use in either model did not affect the outcome.

TABLE 4
Associations Between CSF AD Biomarker Levels (Aβ42 Level,
Ratio Of T-Tau/Aβ42) and Plasma AD Biomarkers

| | |
|---|---|
| Regression | |
| coefficients | p |

Aβ42

| | | |
|---|---|---|
| Male | 4.30 | 0.415 |
| Age | 0.66 | 0.090 |
| No. of APOE4 alleles | −45.47 | <0.001 |
| BNP | −27.99 | <0.001 |
| Pancreatic polypeptide | −18.90 | 0.007 |

t-Tau/Aβ42

| | | |
|---|---|---|
| Male | 0.116 | 0.04 |
| Age | 0.001 | 0.711 |
| No. of APOE4 alleles | 0.297 | <0.001 |
| Pancreatic polypeptide | 0.180 | 0.015 |

Table 4 provides results from linear regression models showing associations between CSF AD biomarker levels (Aβ42 level, ratio of t-tau/Aβ42) and plasma AD biomarkers in all ADNI subjects with CSF and plasma analytes (n is 566). Similar correlations were observed in ADNI subjects with CSF and plasma analytes not treated with cholinesterase inhibitors.

A major roadblock in the identification of candidate biomarkers through previous attempts at multi-analyte profiling has been the successful replication of 1 study's “hits” in other studies. There are many reasons for this, including preanalytical variables, different analytical platforms (such as 2D gel electrophoresis and Luminex multiplexing, among others), and lack of platform cross-validation, body fluid types (plasma, serum), subject selection, disease endophenotypes, and analytical algorithms.

In the Examples described herein, all 3 cohorts (Penn, WU, ADNI) had the same platform, body fluid type, and analytical approaches, with similar methods for subject selection and clinical characterization. Some analytes were previously identified in the multicenter Texas Alzheimer's Research Consortium (TARC) study using the same platform but a different biofluid (serum). See

O'Bryant, et al. A blood-based screening tool for Alzheimer's disease that spans serum and plasma: findings from TARC and ADNI. PloS one, 2011, 6:e28092. The difference in results between a plasma-based study and a serum-based study may be due to protein—protein interactions between analytes of interest and clotting factors, or differential interaction between analytes of interest, additives (e.g., EDTA, serum separation substrate), and potentially different plastic used in the construction of plasma (“purple top”) and serum (“gold top”) tubes. Plasma was collected in tubes containing EDTA in part due to the indeterminate interaction between other additives (e.g., heparin, clot activator) and our analytes of interest.

Beyond fluid type, the analytical platform likely contributes to the lack of overlap between the current study and one prior study using the same biofluid (plasma) but a different platform. Ray, et al. Classification and prediction of clinical Alzheimer's diagnosis based on plasma signaling proteins. See Nat. Med. 2007; 13:1359-1362.

Finally, the use of IUT may generate conservative estimates of overlap, but analytes with the highest likelihood of replication for further technical refinement were focused on. Relaxed IUT can be used to generate common lists, but any algorithm that biases discovery will increase the likelihood of type I error. However, the relatively higher degree of “biomarker concordance” between studies using the same platform would support that future discovery-type studies should be mindful of analytical platform selection as well as analyte identity, and analyte identity and levels should be additionally confirmed by independent means.

### Example 4

### Representative Models

In this example, the analytes to be detected or quantified include one or more of: apoE, BNP, CRP, pancreatic polypeptide, cortisol, E-selectin, Fas, IL-3, IL-13, osteopontin, resistn, stem cell factor.

Statistical Analysis:

In one model, analytes listed above as well as age and gender are analyzed using an artificial neural network designed to maximize sensitivity as a screening test. Analyte values are log-transformed to achieve normal distribution. In addition, Z-transformation was applied to 6 analytes (CRP, E selectin, Fas, IL-3, IL-13, and pancreatic polypeptide) due to batch-to-batch variations in results. The dataset was divided into a training cohort (70%) and a validation cohort (30%). The training cohort was analyzed using the neural network, again with 70% training and 30% validation to establish the most optimal algorithm, and then the algorithm was applied to the original validation cohort. An AD-score was created for each subject, and an optimal cut-off was established to maximize sensitivity for detection. This was able to achieve 88% sensitivity to detect Alzheimer's disease, 75% sensitivity to detect mild cognitive impairment (MCI), with false positive identification of healthy seniors at 39%. Prospectively analyzed biomarkers are then applied to this neural network to determine the AD score to represent the likelihood of each new subject having AD.

In another model, analytes listed above as well as age and gender are analyzed using support vector machine designed to maximize sensitivity. In this model, analyte levels are represented as a function of age and gender, and an optimal division to maximize sensitivity for AD and MCI is created. Each analyte is added to the existing space, and a hyperplane is eventually created as the classification cut-off for a high dimensional space. Prospectively analyzed biomarkers are then applied to this high dimensional space to determine whether it falls on the AD side of the hyperplane or the non-AD side of the hyperplane. The likelihood of each prospectively recruited subject having AD is determined by the relative distance to the classification hyperplane (increasing likelihood with increasing distance from the hyperplane).

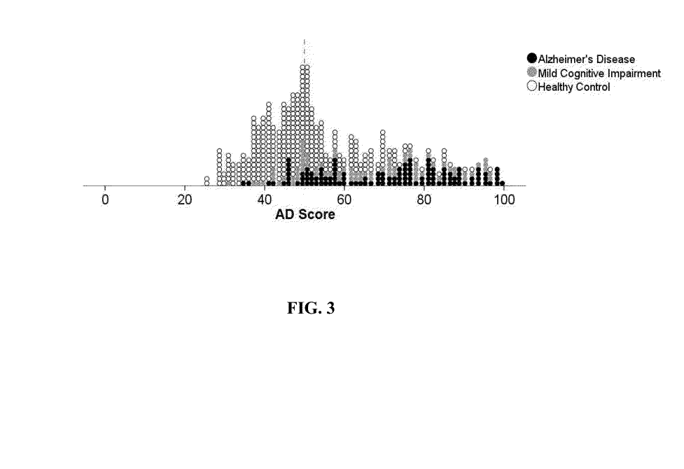

FIG. 3 shows a graphical representation of AD scores. The score is calculated from 1) pseudo-probability of MCI/AD by measuring or detecting the following biomarkers: apoE, BNP, CRP, pancreatic polypeptide, cortisol, E-selectin, Fas, IL-3, IL-13, osteopontin, resistn, and stem cell factor, 2) deriving an optimal cut-off to maximize sensitivity, and 3) transforming the cut-off probability and maximal probability to a scale from 50-100, with score less than 50 represent low risk. Scores greater than 50 represent high risk for AD/MCI.

This disclosure is not limited to particular embodiments described, and as such may vary. The terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of the present disclosure.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the disclosure employ, unless otherwise indicated, techniques of medicine, organic chemistry, biochemistry, molecular biology, pharmacology, and the like, which are within the skill of the art. Such techniques are explained fully in the literature.

As used in the specification and claims, the singular forms “a,” “an,” and “the” include plural referents unless the context clearly dictates otherwise. Thus, for example, reference to “a support” includes a plurality of supports.

We claim:

1. A method of determining if a subject has Alzheimer's disease (AD) or Mild Cognitive Impairment (MCI) or is at risk of developing AD or MCI comprising
(a) measuring the protein levels of Apolipoprotein E (apoE); B-type natriuretic peptide; C-reactive protein; and pancreatic polypeptide in a blood sample from the subject
(b) optionally measuring the protein level Cortisol, FAS, IL-3, IL-10, IL-12p40, IL-13, IL-15, Osteopontin, Resistin, Stem cell factor, E-selectin, serum amyloid protein, or any combination thereof in the blood sample from the subject; and
(c) determining that the subject has Alzheimer's disease (AD) or Mild Cognitive Impairment (MCI), or is at risk of developing AD or MCI when one or more of the following first conditions:
(i) the level of apoE in the subject's blood sample is reduced relative to an apoE control value;
(ii) the level of B-type natriuretic peptide in the subject's blood sample is increased relative to a B-type natriuretic peptide control value;
(iii) the level of C-reactive protein in the subject's blood sample is reduced relative to a C-reactive protein control value;
(iv) the level of pancreatic polypeptide in the subject's blood sample is increased relative to a pancreatic polypeptide control value;
and optionally one or more of the second conditions:
(i) the level of Cortisol in the subject's blood sample is increased relative to a Cortisol control value;
(ii) the level of FAS in the subject's blood sample is increased relative to a FAS control value;
(iii) the level of IL-3 protein in the subject's blood sample is increased relative to an IL-3 control value;
(iv) the level of IL-10 in the subject's blood sample is increased relative to an IL-10 control value;
(v) the level of IL-12p40 in the subject's blood sample is increased relative to an IL-12p40 control value;
(vi) the level of IL-13 in the subject's blood sample is increased relative to an IL-13 control value;
(vii) the level of IL-15 in the subject's blood sample is increased relative to an IL-15 control value;
(viii) the level of Osteopontin in the subject's blood sample is increased relative to an Osteopontin control value;
(ix) the level of Resistin in the subject's blood sample is increased relative to a Resistin control value;
(x) the level of Stem cell factor in the subject's blood sample is increased relative to a Stem cell factor control value;
(xi) the level of E-selectin in the subject's blood sample is reduced relative to an E-selectin control value;
(xii) the level of serum amyloid protein in the subject's blood sample is reduced relative to a serum amyloid protein control value are met.

2. The method of claim 1 wherein two or more of the first conditions are met.

3. The method of claim 2 wherein three or more of the first conditions are met.

4. The method of claim 3 wherein all four of the first conditions are met.

5. The method of claim 4 wherein one or more of the second conditions are met.

6. The method of claim 5 wherein two or more of the second conditions are met.

7. The method of claim 6 wherein three or more of the second conditions are met.

8. The method of claim 7 wherein four or more of the second conditions are met.

9. The method of claim 8 wherein five or more of the second conditions are met.

10. The method of claim 1 wherein the method has a sensitive for determining if a subject has or is at risk of developing AD or MCI of at least 70%.

11. The method of claim 1 wherein the blood sample is whole blood.

12. The method of claim 1 wherein the blood sample is serum.

13. The method of claim 1 wherein the blood sample is plasma.

14. The method of claim 1 wherein the protein levels are measured with an immunoassay.

15. The method of 14 wherein the immunoassay is bead-based assay.

16. The method of claim 15 wherein the immunoassay is a multiplex assay.

17. The method of claim 1 wherein (c) is carried out using a computational system.

18. The method of claim 1 wherein the protein levels are determined to be increased or decreased when the p value between the protein level and the corresponding control value is less than 0.1.

19. The method of claim 18 wherein the protein levels are determined to be increased or decrease when the p value between the measured protein level and the corresponding control value is less than 0.05.

20. The method of claim 19 wherein the protein levels are determined to be increased or decrease when the p value between the measured protein level and the corresponding control value is less than 0.01.

21. The method of claim 1 wherein each control value is the measurement of the corresponding protein level in a blood sample from a control subject that does not have AD or MCI, or an average value for two or more control subjects that do not have AD or MCI.

22. The method of claim 21 wherein the control subjects score 25 or greater on the mini-mental state examination (MMSE).

23. The method of claim 21 wherein the control subjects were determined not to have AD or MCI based on measuring β-amyloid 1-42 (Aβ42), total tau (t-tau), t-tau/Aβ42 ratio or combination thereof in cerebral spinal fluid.

24. A method of determining the efficacy of a treatment for AD or MCI comprising
(a) measuring the protein levels of Apolipoprotein E (apoE); B-type natriuretic peptide; C-reactive protein; and pancreatic polypeptide in a second blood sample from a subject with AD or MCI undergoing a treatment for AD or MCI, wherein the second blood sample is obtained from the subject after a sufficient amount of time has passed for the treatment to reduce one or more symptoms of the AD or MCI;
(b) optionally measuring the protein level of Cortisol, FAS, IL-3, IL-10, IL-12p40, IL-13, IL-15, Osteopontin, Resistin, Stem cell factor, E-selectin, serum amyloid protein, or any combination thereof in the second blood sample from the subject;
(c) determining that the treatment is effective for treating AD or MCI when one or more of the following first conditions:
(i) the level of apoE in the subject's second blood sample is increased relative to a first blood sample taken from the subject prior the treatment;
(ii) the level of B-type natriuretic peptide in the subject's second blood sample is decreased relative to a first blood sample taken from the subject prior the treatment;
(iii) the level of C-reactive protein in the subject's second blood sample is increased relative to a first blood sample taken from the subject prior the treatment;
(iv) the level of pancreatic polypeptide in the subject's second blood sample is decreased relative to a first blood sample taken from the subject prior the treatment;
and optionally one or more of the second conditions:
(i) the level of Cortisol in the subject's second blood sample is decreased relative to a first blood sample taken from the subject prior the treatment;
(ii) the level of FAS in the subject's second blood sample is decreased relative to a first blood sample taken from the subject prior the treatment;
(iii) the level of IL-3 protein in the subject's second blood sample is decreased relative to a first blood sample taken from the subject prior the treatment;
(iv) the level of IL-10 in the subject's second blood sample is decreased relative to a first blood sample taken from the subject prior the treatment;
(v) the level of IL-12p40 in the subject's second blood sample is decreased relative to a first blood sample taken from the subject prior the treatment;
(vi) the level of IL-13 in the subject's second blood sample is decreased relative to a first blood sample taken from the subject prior the treatment;
(vii) the level of IL-15 in the subject's second blood sample is decreased relative to a first blood sample taken from the subject prior the treatment;
(viii) the level of Osteopontin in the subject's second blood sample is decreased relative to a first blood sample taken from the subject prior the treatment;
(ix) the level of Resistin in the subject's second blood sample is decreased relative to a first blood sample taken from the subject prior the treatment;
(x) the level of Stem cell factor in the subject's second blood sample is decreased relative to a first blood sample taken from the subject prior the treatment;
(xi) the level of E-selectin in the subject's second blood sample is increased relative to a first blood sample taken from the subject prior the treatment;
(xii) the level of serum amyloid protein in the subject's second blood sample is increased relative to a first blood sample taken from the subject prior the treatment are met.

25. The method of claim 24 wherein all four for the first conditions and 3 or more of the second conditions are met.

26. A method comprising the steps of,
a) measuring a blood sample from a subject for levels of the following proteins Apolipoprotein E (apoE); B-type natriuretic peptide; C-reactive protein; and pancreatic polypeptide providing measured levels;
b) comparing the normalized measured levels of proteins with reference levels wherein the reference levels are obtained from normalized measured values;
c) determining whether the subject is at increased risk of Alzheimer's disease (AD) and Mild Cognitive Impairment (MCI);
wherein if the subject has altered levels of the proteins compared to reference levels this indicates an increased risk of Alzheimer's disease (AD) or Mild Cognitive Impairment (MCI).

27. The method of claim 26, further comprising the steps of analyzing at least five more the following proteins, Cortisol, E-selectin, FAS, Gamma-IFN-induced monokine, IL-3, IL-10, IL-12p40, IL-13, IL-15, Osteopontin, Resistin, Serum amyloid protein, and Stem cell factor; providing measured levels of the proteins.

28. The method of claim 27, further comprising the steps of testing the subject for β-amyloid 1-42 (Aβ42), total tau (t-tau) and t-tau/Aβ42 ratio from a CSF sample provided from the subject is indicated to have an increased risk of Alzheimer's disease (AD) or Mild Cognitive Impairment (MCI) based on the measured levels of the proteins.

29. The method of claim 27, whereby the diagnosis of AD is aided by determining a difference between the normalized measured levels of proteins to the reference levels of the protein from non-AD samples wherein the difference meets or exceeds a statistically significant difference between normalized measured values of proteins in the blood samples from individuals without AD and individuals with AD, wherein the statistically significant difference indicates a diagnosis of AD.

30. The method of claim 26 for diagnosing or monitoring the progression of AD or MCI by obtaining a measured value for ApoE, BNP, CRP, and pancreatic polypeptide in blood sample; and comparing said measure value of ApoE, BNP, CRP, and pancreatic polypeptide with a reference value; wherein the measured level of BNP and Pancreatic polypeptide increases, wherein the measured levels of ApoE and CRP decrease indicates a diagnosis or the progression of MCI or AD.

31. The method of claim 27 that further comprises comparing measured values from blood samples for BNP, Cortisol, FAS, IL-3, IL-10, IL-12p40, IL-13, IL-15, Osteopontin, Pancreatic polypeptide, Resistin, Stem cell factor, ApoE, CRP, E-selectin, and serum amyloid protein with reference values for BNP, Cortisol, FAS, IL-3, IL-10, IL-12p40, IL-13, IL-15, Osteopontin, Pancreatic polypeptide, Resistin, Stem cell factor, ApoE, CRP, E-selectin, and serum amyloid protein, wherein measured values are from individuals with an MMSE score less than 25, wherein the measured value for ApoE, CRP, E-selectin, and serum amyloid protein decreases, wherein measured values for BNP, Cortisol, FAS, IL-3, IL-10, IL-12p40, IL-13, IL-15, Osteopontin, Pancreatic polypeptide, Resistin, Stem cell factor increase indicating cognitive impairment such as MCI or AD.

32. The method of claim 27, wherein the subject is a human subject seeking a diagnosis for AD.

33. The method of claim 27, wherein the sample is a whole blood, plasma or serum.

34. The method of claim 27, wherein the measuring comprises mixing the sample with a solid surface comprising a ligand or capture antibody to the protein and detecting the protein bound to the surface.

35. The method of claim 27, wherein the reference levels for the proteins are obtained by a method comprising: determining the mean value of the normalized measured levels of the protein biomarkers in normal individuals with Mini Mental State examination (MMSE) scores from 25-30, having statistically significant difference from the mean value of the normalized measured levels of the proteins from a subjects with MMSE score of lower than 24.

36. The method of claim 27 wherein the significant difference in the normalized measured values of the 17 protein biomarkers in the blood samples from individuals with AD in comparison to samples from individuals without AD is calculated using Significance Analysis of Microarrays (SAM).

37. A kit comprising at least one reagent specific for at least one protein selected from the group consisting of proteins in claim 27 and instructions for carrying out the method of claim 27.

38. The kit of claim 37, wherein the reagent is specific for at least four, five, six, seven, eight, nine, ten, eleven, twelve, thirteen, fourteen, fifteen or more proteins selected from the group consisting of Apolipoprotein E (apoE); B-type natriuretic peptide; C-reactive protein; pancreatic polypeptide, cortisol, FAS, IL-3, IL-10, IL-12p40, IL-13, IL-15, Osteopontin, Resistin, Stem cell factor, E-selectin, serum amyloid protein, or any combination.

39. The kit of claim 37, wherein the reagent specific protein is an antibody, or fragment thereof, that is specific for said protein.

40. A surface comprising attached thereto, at least one reagent specific for each protein as provided in claim 27.

41. The surface of claim 40, further comprising the reagent bound to the protein and a secondary reagent specific for the protein bound to the protein wherein the secondary agent comprises a marker.

42. The surface of claim 41, wherein the marker is a fluorescent molecule or reporter.

43. A computer readable format comprising the values obtained by the method of claim 15.

44. A system for detecting proteins of 27, comprising a solid surface of claim 40 and a visualization device.
